(19) United States
(12) Patent Application Publication
(10) Pub. No.: US 2025/0281126 A1
(43) Pub. Date: Sep. 11, 2025

(54) ON-DEMAND OVERNIGHT HYPO RISK DETECTOR

(71) Applicant: Abbott Diabetes Care Inc., Alameda, CA (US)

(72) Inventors: Gary A. HAYTER, Oakland, CA (US); Taihao JIN, Richmond, CA (US); Tao PENG, Alameda, CA (US); Erwin S. BUDIMAN, Fremont, CA (US); Matthew T. NOVAK, Oakland, CA (US)

(21) Appl. No.: 19/072,885

(22) Filed: Mar. 6, 2025

Related U.S. Application Data
(60) Provisional application No. 63/562,190, filed on Mar. 6, 2024.

(51) Int. Cl. A61B 5/00; A61B 5/145

(52) U.S. Cl. CPC A61B 5/7275; A61B 5/14532; A61B 5/7267; A61B 5/742; A61B 5/748

(57) ABSTRACT

Disclosed herein are system, method, and computer program product embodiments for generating determining overnight hypoglycemia risk by estimating the likelihood of a hypoglycemic event occurring over a specified period of time, namely overnight. The disclosure describes utilizing two key aspects: factors that can disturb glucose levels are much less likely to occur overnight, and bedtime is a convenient and beneficial time for the patient to check for and mitigate their risk of hypoglycemia overnight.

### CROSS-REFERENCE TO RELATED APPLICATIONS

This application claims priority to U.S. provisional application 63/562,190, filed on Mar. 6, 2024, the entirety of which is hereby incorporated by reference.

### FIELD OF THE INVENTION

This disclosure generally relates to continuous analyte sensors, including in vivo analyte sensors for sensing and monitoring analytes in a bodily fluid, and the use of such sensors for detecting on-demand overnight hypoglycemic risks of users.

### BACKGROUND

Prior art methods for predicting future hypoglycemia occurrence can include continuously running, real-time systems that can estimate glucose levels at a future elapsed time such as 15 minutes, 60 minutes, or 90 minutes. However, generally this prediction has a high degree of uncertainty, especially as the time horizon is increased (e.g., 2 hours or more) because many unknown factors such as eating, activity, stress, etc., can alter the trajectory of glucose levels after the time the prediction is made. The result is that prior art hypoglycemia predictors are unreliable at predicting glucose levels over an extended time horizon, such as an overnight period, which can result in frequent false positive alerts and reduce the accuracy and usefulness of the overnight prediction.

### SUMMARY

Accordingly, the present disclosure describes an improved risk detection model for predicting glucose levels over an extended time horizon including periods where patients are most vulnerable and when hypoglycemia prediction is most valuable to a patient, e.g., the overnight period. Another aspect of the present disclosure is the ability to provide estimates regarding the likelihood of a hypoglycemic event occurring over a specified period of time, namely overnight, rather than predicting the glucose level at a specific future elapsed time. The overnight period can be predetermined or user defined, such as from 10 P.M. to 7 A.M., or from the time that the detection model is activated through a predefined end time, e.g., 7 A.M.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1A is a system overview of a sensor applicator, data receiving device, monitoring system, network, and remote system.

FIG. 1B is a diagram illustrating an operating environment of an example analyte monitoring system for use with the techniques described herein.

FIG. 2A is a block diagram depicting an example embodiment of a data receiving device.

FIG. 2B is a block diagram illustrating an example data receiving device for communicating with the sensor according to exemplary embodiments of the disclosed subject matter.

FIGS. 2C and 2D are block diagrams depicting example embodiments of sensor control devices.

FIG. 2E is a block diagram illustrating an example analyte sensor according to exemplary embodiments of the disclosed subject matter.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 4D is a top perspective view of an exemplary applicator device in accordance with the disclosed subject matter.

FIG. 4E is a bottom perspective view of the applicator device of FIG. 4D.

FIG. 4F is an exploded view of the applicator device of FIG. 4D.

FIG. 4G is a side cutaway view of the applicator device of FIG. 4D.

FIG. 5 is a diagram illustrating example operational states of the sensor according to exemplary embodiments of the disclosed subject matter.

FIG. 6 is a diagram illustrating an example operational and data flow for over-the-air programming of a sensor according to the disclosed subject matter.

FIG. 7 is a diagram illustrating an example data flow for secure exchange of data between two devices according to the disclosed subject matter.

FIG. 8 is a diagram illustrating a system for implementing a machine learning prediction model(s) for generating overnight hypo risk predictions and updating potential actions in response to hypo risk predictions.

FIG. 9 is a flowchart illustrating the method of updating graphical user interfaces based on hypo risk predictions.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for an improved risk prediction model for predicting glucose levels over an extended time horizon. The risk prediction model can be deployed for providing recommendations and suggestions to patients based on any predicted risk, including hypoglycemia. The disclosure described herein addresses a key scenario where a patient is most vulnerable to hypoglycemia and enables a practical means for the patient to mitigate the risk. The overnight period, and in particular the early morning period, is when the patient is most vulnerable to hypoglycemia because they are typically asleep at this time and severe hypoglycemia can occur when the patient is unaware. This disclosure exploits two key aspects: a) factors that can disturb glucose levels are much less likely to occur overnight, and b) bedtime is a convenient and beneficial time for the patient to check for and mitigate their risk of hypoglycemia overnight.

Generating accurate hypoglycemia predictions for extended time horizons that works during the daytime is challenging because there are often unmeasured disturbances or events that contribute to the patient's glucose at this time. After the patient has gone to sleep, the frequency and degree of impact of these disturbances greatly decreases, and therefore the predictability of hypoglycemia risk is greater and the frequency of false positive hypoglycemia risk alerts will be less. The present disclosure capitalizes on these factors by training a model to address predictions over the extended time horizons.

Complications from patient activities and other possible impactful events during the day when patients are awake can reduce the accuracy of glucose level predictions. This reduced accuracy can cause predictive hypoglycemia alerts to be less useful because of the high frequency of false positives and because patients can often respond quickly to non-predictive hypoglycemia alerts at this time. Bedtime is often an ideal time for patients to check for their risk of future hypoglycemia, or “hypo risk.” The patient is performing activities for getting ready for bed and checking for overnight hypo risk would be just one more activity. The present disclosure utilizes an improved risk detection model for generating predictions and providing notifications and/or recommendations (e.g. in the form of visual elements and particularly textual elements on a graphical user interface) based on any predicted overnight hypo risk, which can allow for the patient to take a mitigating action before they go to sleep. For example, the risk detection model can detect or predict an overnight risk based on a glucose level prediction over an extended time horizon (rather than a particular moment in time).

When the risk detection model detects (or predicts) an overnight hypo risk, the risk model can identify and provide visual elements (e.g. textual elements) indicating mitigating actions, such as to a graphical user interface that is updated based on the overnight prediction and optionally to a personalized graphical user interface that is additionally updated based on user-specific information (e.g., an evolving medical user interface). Examples of such mitigating actions include consuming food or some form of nutrition, what type of food or nutrition to consume, setting the low glucose alarm (i.e. an alarm that is triggered when glucose is below a predetermined threshold, e.g. 56 mg/dL, 70 mg/dL, and the like), setting the predicted low glucose alarm (i.e. an alarm that is triggered when glucose is predicted to be below the predetermined threshold). The risk detection model can be implemented in an application that can be configured to be installed on a user's device, such as a mobile device. The application can provide configurable options to turn off alarms automatically at some time in the morning. In some embodiments, the application is a continuous glucose monitoring (CGM) application.

In some embodiments, the CGM application modifies the graphical user interface to display visual elements (e.g. textual elements) including the overnight hypo risk prediction. In this way, the CGM application can dynamically display information as needed for a user that is prepared to take the appropriate mitigation action to address the identified hypo risk. In an alternative embodiment, the risk detection model is configured to operate continuously (e.g., every time a new glucose reading is available which can occur at a predefined or predetermined cadence such as once a minute) and to control the CGM application to provide a notification or alert if overnight hypo risk is detected.

The risk detection model is enabled or selected responsive to a determination that an upcoming time period corresponds to a future overnight time period. In some embodiments, determination that the upcoming time period corresponds to the future overnight time period can be based on a user input received via the graphical user interface. In some embodiments, the risk detection model can be enabled at a predetermined time associated with determining an overnight hypo risk such as a prespecified time period, such as 6 pm-10 pm or at a specific time of day, such as 10 pm. The predetermined time can be associated with a time period that is determined to provide more accurate predictions for overnight hypo risk, and can be determined via a schedule (e.g., provided by a trained scheduling model) and/or configured by the user. For example, the schedule provide, as an output, a recommended time for the risk detection model to run and can receive, as an input, user medical history for a predetermined time period (e.g., over the past week), user activity for that day or another predetermined time period (e.g., over the past 3 days), and user dosage history.

Before the present subject matter is described in detail, it is to be understood that this disclosure is not limited to the particular embodiments described, as such can, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided can be different from the actual publication dates which can need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor subsystem and a sharp subsystem contained therein. The user can couple the sensor subsystem to the electronics housing, and can couple the sharp to the applicator with an assembly process that involves the insertion of the applicator into the container in a specified manner. In other embodiments, the applicator, sensor control device, sensor subsystem, and sharp subsystem can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

As mentioned, a number of embodiments of systems, devices, and methods are described herein that provide for the improved assembly and use of dermal sensor insertion devices for use with in vivo analyte monitoring systems. In particular, several embodiments of the present disclosure are designed to improve the method of sensor insertion with respect to in vivo analyte monitoring systems and, in particular, to prevent the premature retraction of an insertion sharp during a sensor insertion process. Some embodiments, for example, include a dermal sensor insertion mechanism with an increased firing velocity and a delayed sharp retraction. In other embodiments, the sharp retraction mechanism can be motion-actuated such that the sharp is not retracted until the user pulls the applicator away from the skin. Consequently, these embodiments can reduce the likelihood of prematurely withdrawing an insertion sharp during a sensor insertion process; decrease the likelihood of improper sensor insertion; and decrease the likelihood of damaging a sensor during the sensor insertion process, to name a few advantages. Several embodiments of the present disclosure also provide for improved insertion sharp subsystems to account for the small scale of dermal sensors and the relatively shallow insertion path present in a subject's dermal layer. In addition, several embodiments of the present disclosure are designed to prevent undesirable axial and/or rotational movement of applicator components during sensor insertion. Accordingly, these embodiments can reduce the likelihood of instability of a positioned dermal sensor, irritation at the insertion site, damage to surrounding tissue, and breakage of capillary blood vessels resulting in fouling of the dermal fluid with blood, to name a few advantages. In addition, to mitigate inaccurate sensor readings which can be caused by trauma at the insertion site, several embodiments of the present disclosure can reduce the end-depth penetration of the needle relative to the sensor tip during insertion.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a data receiving device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a data receiving device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

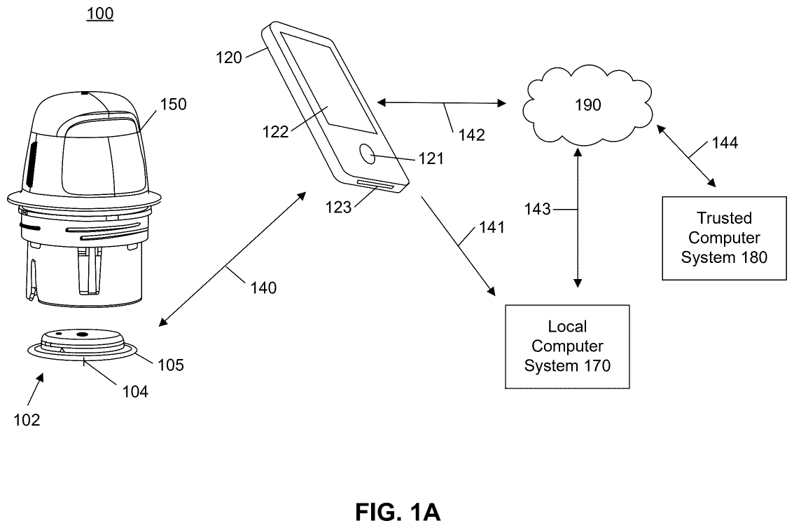

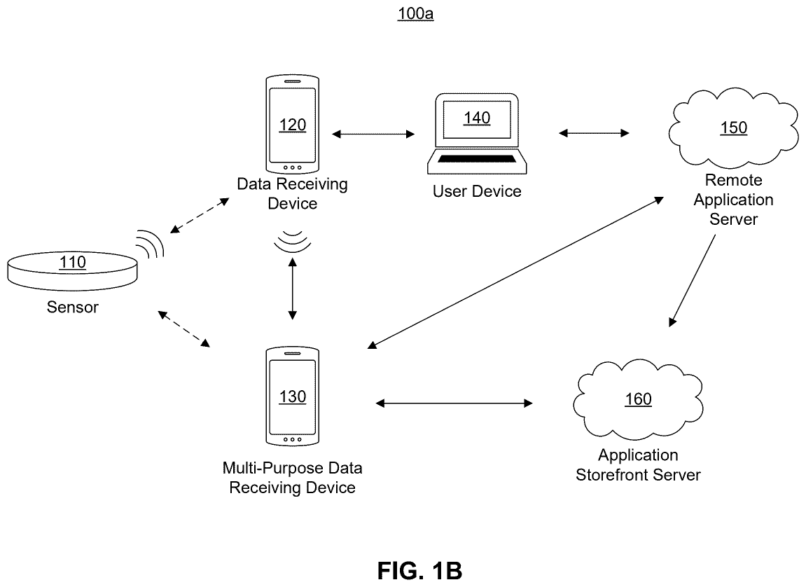

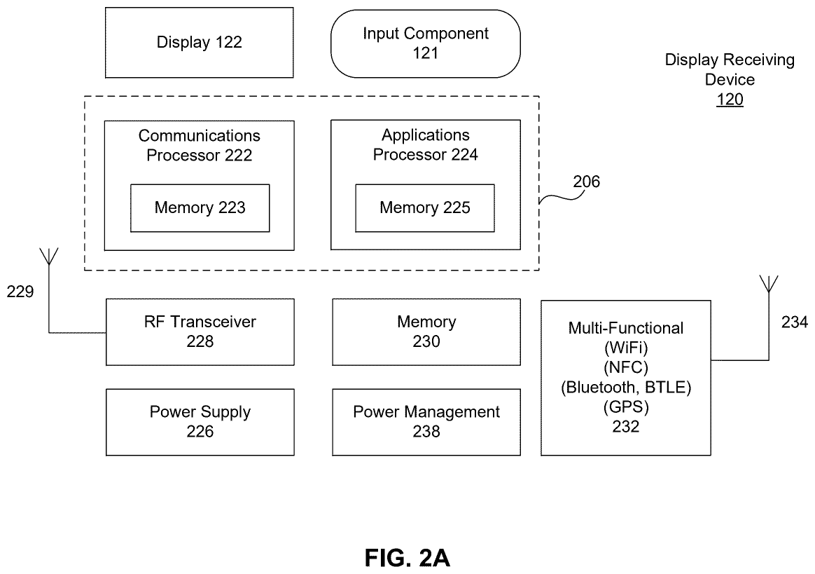

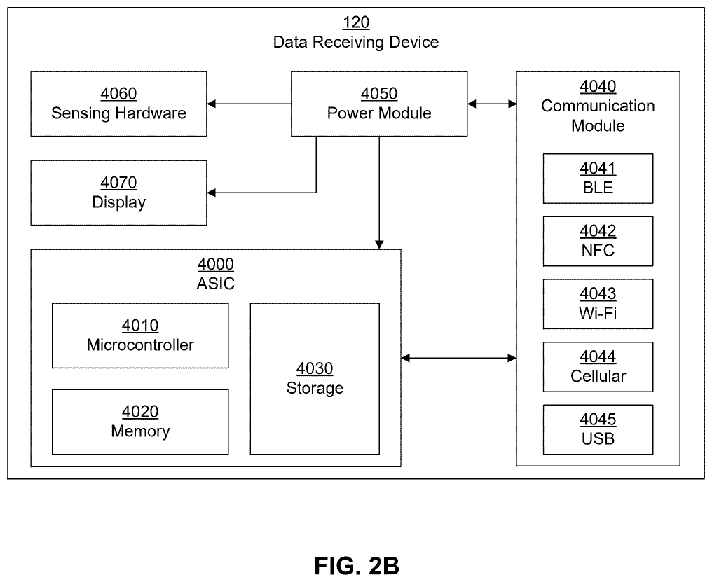

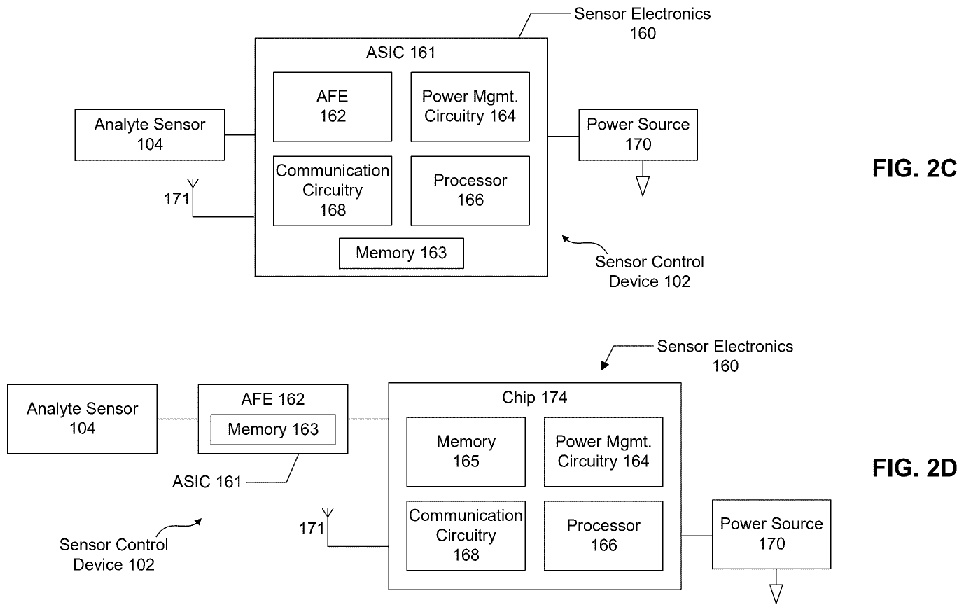

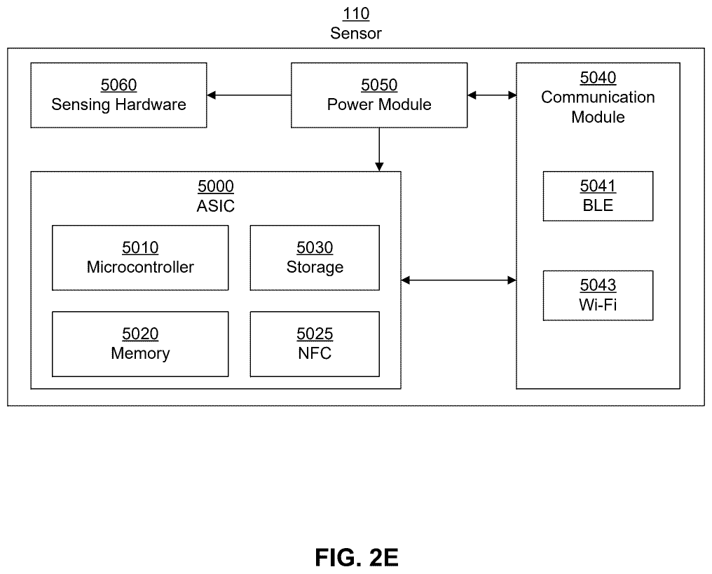

FIG. 1A is a conceptual diagram depicting an example embodiment of an analyte monitoring system 100 that includes applicator device 150, sensor control device 102, and data receiving device 120. Here, applicator device 150 can be used to deliver sensor control device 102 to a monitoring location on a user's skin where in vivo analyte sensor 104 is maintained in position for a period of time by adhesive patch 105. Sensor control device 102 is further described in FIGS. 2B and 2C, and can communicate with data receiving device 120 via a communication path 141 using a wired or wireless technique. Example wireless protocols include Bluetooth, Bluetooth Low Energy (“BLE” or “BTLE”), Bluetooth SMART, NFC, and others. Users can monitor applications installed in memory on data receiving device 120 using display 122 and input component 121 and the device battery can be recharged using power port 123. More detail about data receiving device 120 is set forth with respect to FIG. 2A below. Data receiving device 120 can communicate with local computer system 175 via a communication path 141 using a wired or wireless technique. Local computer system 175 can include one or more of a laptop, desktop, tablet, phablet, smartphone, set-top box, video game console, or other computing device and wireless communication can include any of a number of applicable wireless networking protocols including Bluetooth, BTLE, Wi-Fi or others. Local computer system 175 can communicate via communications path 143 with a network 190 similar to how data receiving device 120 can communicate via a communications path 142 with network 190, by wired or wireless technique as described previously. Network 190 can be any of a number of networks, such as private networks and public networks, local area or wide area networks, a cloud network, and so forth. A trusted computer system 180, which can be a cloud-based system, can include a server and can provide authentication services and secured data storage and can communicate via communications path 144 with network 190 by wired or wireless technique. In some embodiments, data receiving device 120 can be implemented as a mobile device, such as a smartphone, tablet, or watch device.

FIG. 1B illustrates an operating environment of analyte monitoring system 100a capable of embodying the techniques described herein. Analyte monitoring system 100a can include a system of components designed to provide monitoring of parameters, such as analyte levels, of a human or animal body or can provide for other operations based on the configurations of the various components. As embodied herein, the system can include analyte sensor 110, or simply “sensor” worn by the user or attached to the body for which information is being collected. As embodied herein, analyte sensor 110 can be a sealed, disposable device with a predetermined active use lifetime (e.g., 1 day, 14 days, 30 days, etc.). Analyte sensor 110 can be applied to the skin of the user body and remain adhered over the duration of the sensor lifetime or can be designed to be selectively removed and remain functional when reapplied. Analyte monitoring system 100a can further include data receiving device 120 or multi-purpose data receiving device 130 configured as described herein to facilitate retrieval and delivery of data, including analyte data, from analyte sensor 110.

As embodied herein, analyte monitoring system 100a can include a software or firmware library or application provided, for example via remote application server 155 or application storefront server 160, to a third-party and incorporated into multi-purpose data receiving device 130 such as a mobile phone, tablet, personal computing device, or other similar computing device capable of communicating with analyte sensor 110 over a communication link. Multi-purpose hardware can further include embedded devices, including, but not limited to insulin pumps or insulin pens, having an embedded library configured to communicate with analyte sensor 110. Although the illustrated embodiments of analyte monitoring system 100a include only one of each of the illustrated devices, this disclosure contemplates analyte monitoring system 100a incorporating multiples of each component interacting throughout the system. For example and without limitation, as embodied herein, data receiving device 120 and/or multi-purpose data receiving device 130 can include multiples of each. As embodied herein, multi-purpose data receiving device 130 can communicate directly with analyte sensor 110 as described herein. Additionally or alternatively, multi-purpose data receiving device 130 can communicate with multi-purpose data receiving device 130 to provide analyte data, or visualization or analysis of the data, for secondary display to the user or other authorized parties.

### Exemplary Data Receiving Device

FIG. 2A is a block diagram depicting an example embodiment of a data receiving device 120 configured as a smartphone. Here, data receiving device 120 can include a display 122, input component 121, and processing core 206 including communications processor 222 coupled with memory 223 and applications processor 224 coupled with memory 225. Also included can be separate memory 230, RF transceiver 228 with antenna 229, and power supply 226 with power management subsystem 238. Further included can be a multi-functional transceiver 232 which can communicate over Wi-Fi, NFC, Bluetooth, BTLE, and GPS with antenna 234. As understood by one of skill in the art, these components are electrically and communicatively coupled in a manner to make a functional device.

Memory 230 can be configured to store the medical application (e.g., CGM application) and a user interface model. Optionally, the trained user interface model can be a machine learning model that can be trained remotely from data receiving device, such as at remote application server 155 or any other server associated with the medical application. The user interface model can be configured to generate proposed user interface modifications to a graphical medical user interface of the medical application installed on data receiving device 120. The user interface model can be implemented as subsystem of the medical application and both can be configured to receive and process medical data including real-time medical data from a medical sensor (e.g., CGM sensor) and historical medical data (e.g., stored on the data receiving device 120 or remotely, such as at a backend database or server). The user interface model and/or the medical application can also be granted access privileges to user interactions with data receiving device 120 that include, for example, other applications installed on the data receiving device 120, duration of user interaction with the data receiving device 120 and/or the other installed applications, and date and time of user interaction with the data receiving device 120 and/or the other installed applications. The user interface model and/or the medical application can also be configured to connect with databases remote from data receiving device 120 that can include historical medical data, such as medical history from an HCP, as well as cohort data that represents anonymized medical data collected from the medical sensors of other users. A cohort can be established for the current user based on one or more similar characteristics to the current user, such as, user age, user medical condition, user medical history, user location, and user preference, just to name a few examples.

### Exemplary Data Receiving Device Architecture

For purpose of illustration and not limitation, reference is made to the exemplary embodiment of data receiving device 120 for use with the disclosed subject matter as shown in FIG. 2B. Data receiving device 120 and the related multi-purpose data receiving device 130 include components germane to the discussion of analyte sensor 110 and its operations and additional components can be included. Data receiving device 120 can also be implemented with components for generating different interface elements of a graphical user interface that is displayed to the user. For example, data receiving device 120 can receive and store a user interface model trained to receive any combination of user analyte data, user medical history, and other user activity data (e.g., device interaction, tracked physical activity, tracked meals) and generate the interface elements for the graphical user interface. In particular embodiments, data receiving device 120 and multi-purpose data receiving device 130 can be or include components provided by a third party and are not necessarily restricted to include devices made by the same manufacturer as analyte sensor 110.

As illustrated in FIG. 2B, data receiving device 120 includes ASIC 4000 having microcontroller 4010, memory 4020, and storage 4030 and communicatively coupled with a communication subsystem 4040. Power for the components of data receiving device 120 can be delivered by power subsystem 4050, which as embodied herein can include a rechargeable battery. Data receiving device 120 can further include display 4070 for facilitating review of analyte data received from analyte sensor 110 or other device (e.g., user device 140 or remote application server 155). Data receiving device 120 can include separate user interface components (e.g., physical keys, light sensors, microphones, etc.).

Memory 4020 can be configured to store a medical application (e.g., CGM application) that is configured to communicate with and receive data from the analyte sensor of the user. The medical application can further be implemented with a trained user interface model, similar to the configuration discussed with regard to FIG. 2A.

Communication subsystem 4040 can include BLE subsystem 4041 and NFC subsystem 4042. Data receiving device 120 can be configured to wirelessly couple with analyte sensor 110 and transmit commands to and receive data from analyte sensor 110. As embodied herein, data receiving device 120 can be configured to operate, with respect to analyte sensor 110 as described herein, as an NFC scanner and a BLE end point via specific subsystems (e.g., BLE subsystem 4042 or NFC subsystem 4043) of communication subsystem 4040. For example, data receiving device 120 can issue commands (e.g., activation commands for a data broadcast mode of the sensor; pairing commands to identify data receiving device 120) to analyte sensor 110 using a first subsystem of the communication subsystem 4040 and receive data from and transmit data to analyte sensor 110 using a second subsystem of the communication subsystem 4040. Data receiving device 120 can be configured for communication with user device 140 via a Universal Serial Bus (“USB”) subsystem 4045 of the communication subsystem 4040.

As another example, communication subsystem 4040 can include, for example, cellular radio subsystem 4044. The cellular radio subsystem 4044 can include one or more radio transceivers for communicating using broadband cellular networks, including, but not limited to third generation (“3G”), fourth generation (“4G”), and fifth generation (“5G”) networks. Additionally, communication subsystem 4040 of data receiving device 120 can include Wi-Fi radio subsystem 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using cellular radio subsystem 4044 or Wi-Fi radio subsystem 4043, data receiving device 120 can communicate with remote application server 155 to receive analyte data or provide updates or input received from a user (e.g., through one or more user interfaces). Although not illustrated, communication subsystem 5040 of analyte sensor 110 can similarly include a cellular radio subsystem or Wi-Fi radio subsystem.

As embodied herein, on-board storage 4030 of data receiving device 120 can store analyte data received from analyte sensor 110. Further, data receiving device 120, multi-purpose data receiving device 130, or user device 140 can be configured to communicate with remote application server 155 via a wide area network. As embodied herein, analyte sensor 110 can provide data to data receiving device 120 or multi-purpose data receiving device 130. Data receiving device 120 can transmit the data to user device 140. User device 140 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor 110. In particular embodiments, data receiving device 120 can be configured to operate in coordination with analyte sensor 110 and based on analyte data received from the analyte sensor 110. As an example, where analyte sensor 110 is a glucose sensor, data receiving device 120 can be or include an insulin pump or insulin injection pen. In coordination, multi-purpose data receiving device 130 can adjust an insulin dosage for a user based on glucose values received from the analyte sensor.

### Exemplary Sensor Control Devices

FIGS. 2C and 2D are block diagrams depicting example embodiments of sensor control device 102 having in vivo analyte sensor 104 and sensor electronics 169 (including analyte monitoring circuitry) that can have the majority of the processing capability for rendering end-result data suitable for display to the user. In FIG. 2C, a single semiconductor chip 161 is depicted that can be a custom application specific integrated circuit (“ASIC”). Shown within ASIC 161 are certain high-level functional units, including an analog front end (“AFE”) 162, power management circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

Memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to data receiving device 120 (not shown), for example, where minimal further processing is needed by the resident software application to display the data.

FIG. 2D is similar to FIG. 2C but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

For purpose of illustration and not limitation, reference is made to the exemplary embodiment of analyte sensor 110 for use with the disclosed subject matter as shown in FIG. 2E. FIG. 2E illustrates a block diagram of an example analyte sensor 110 according to exemplary embodiments compatible with the security architecture and communication schemes described herein.

As embodied herein, analyte sensor 110 can include ASIC 5000 communicatively coupled with a communication subsystem 5040. ASIC 5000 can include a microcontroller core 5010, on-board memory 5020, and storage memory 5030. Storage memory 5030 can store data used in an authentication and encryption security architecture. Storage memory 5030 can store programming instructions for analyte sensor 110. As embodied herein, certain communication chipsets can be embedded in the ASIC 5000 (e.g., an NFC transceiver 5025). ASIC 5000 can receive power from a power subsystem 5050, such as an on-board battery or from an NFC pulse. Storage memory 5030 of the ASIC 5000 can be programmed to include information such as an identifier for analyte sensor 110 for identification and tracking purposes. Storage memory 5030 can also be programmed with configuration or calibration parameters for use by analyte sensor 110 and its various components. Storage memory 5030 can include rewritable or one-time programming (“OTP”) memory. The storage memory 5030 can be updated using techniques described herein to extend the usefulness of analyte sensor 110.

As embodied herein, communication subsystem 5040 of sensor 110 can be or include one or more subsystems to support analyte sensor 110 communicating with other devices of the analyte monitoring system 100. As an example only and not by way of limitation, example communication subsystems 5040 can include BLE subsystem 5041 As used throughout this disclosure, BLE refers to a short-range communication protocol optimized to make pairing of Bluetooth devices simple for end users. Communication subsystem 5040 can transmit and receive data and commands via interaction with similarly-capable communication subsystems of data receiving device 120 or user device 140. Communication subsystem 5040 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, sensor 110 can further include suitable sensing hardware 5060 appropriate to its function. As embodied herein, sensing hardware 5060 can include an analyte sensor transcutaneously or subcutaneously positioned in contact with a bodily fluid of a subject. The analyte sensor can generate sensor data containing values corresponding to levels of one or more analytes within the bodily fluid.

### Exemplary Assembly Processes for Sensor Control Devices

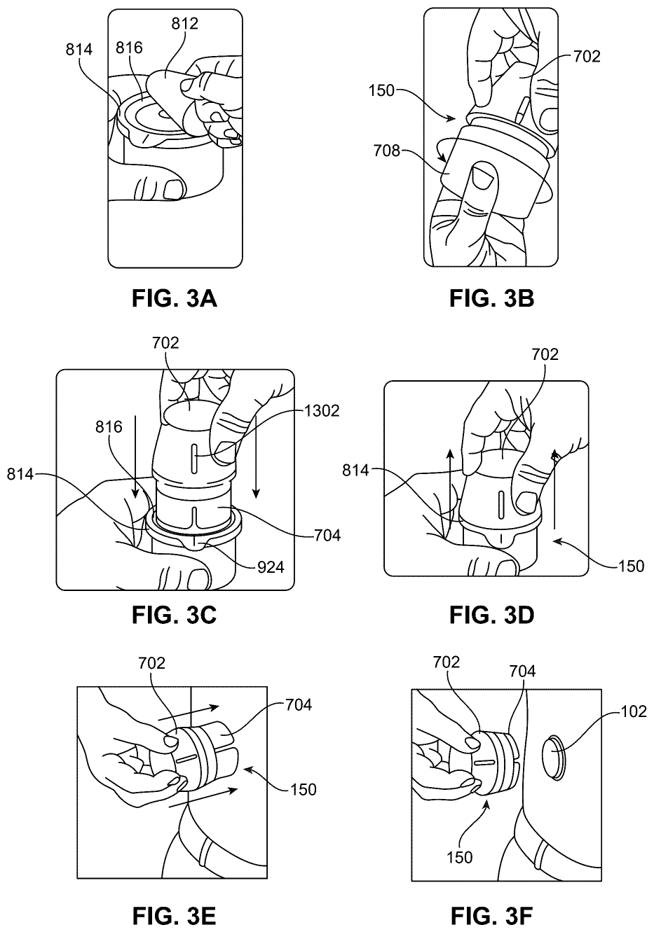

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of user preparing tray 810, configured here as a tray (although other packages/containers can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting applicator device 150 into tray 810 during an assembly. Initially, the user can insert sheath 704 into platform 808 inside tray 810 after aligning a housing orienting feature (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from tray 810 during an assembly. A user can remove applicator 150 from tray 810 by proximally advancing housing 702 with respect to tray 810 or other motions having the same end effect of uncoupling applicator 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator 150 from the application site.

Analyte monitoring system 100, described with respect to FIGS. 3A-3F and elsewhere herein, can provide a reduced or eliminated chance of accidental breakage, permanent deformation, or incorrect assembly of applicator components compared to prior art systems. Since housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Exemplary Sensor Applicator Devices

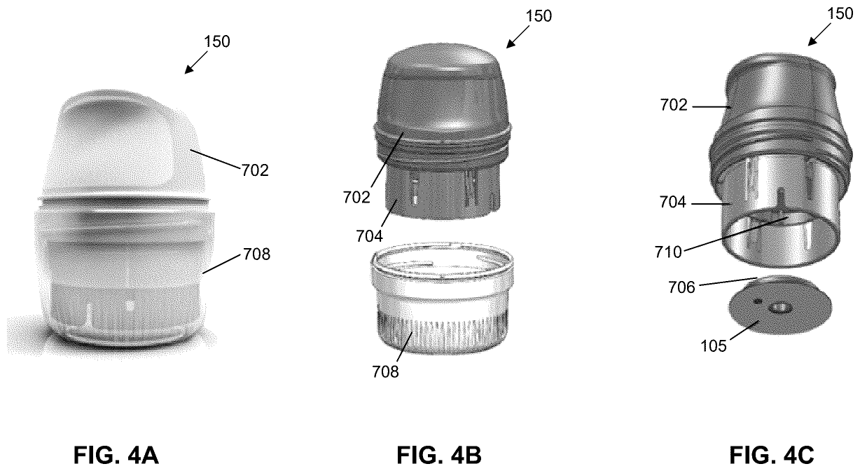

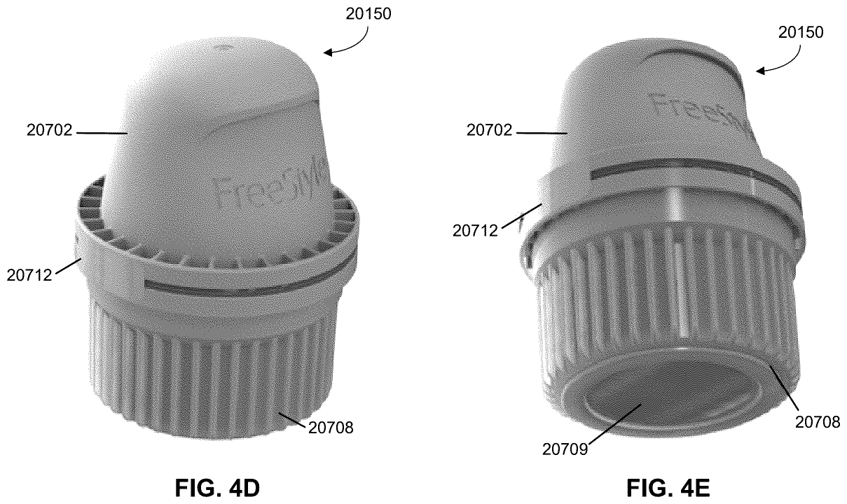

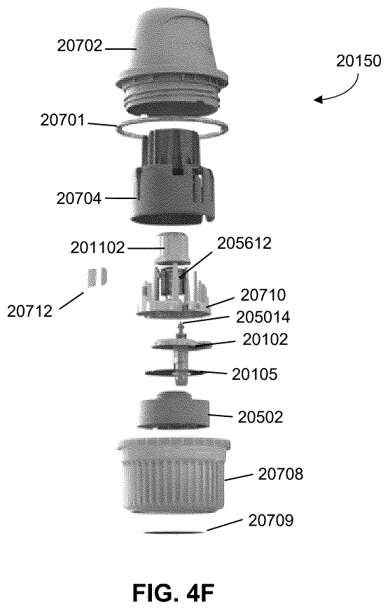

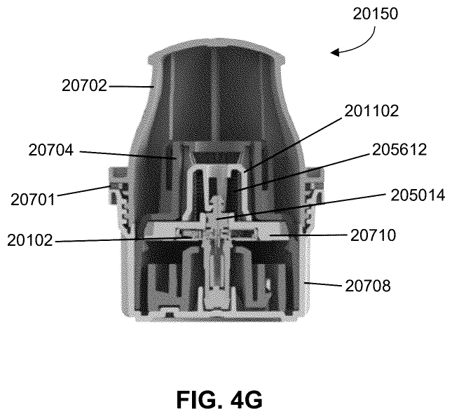

FIG. 4A is a side view depicting an example embodiment of an applicator device 150 coupled with screw cap 708. This is an example of how applicator 150 is shipped to and received by a user, prior to assembly by the user with a sensor. FIG. 4B is a side perspective view depicting applicator 150 and cap 708 after being decoupled. FIG. 4C is a perspective view depicting an example embodiment of a distal end of applicator device 150 with electronics housing 706 and adhesive patch 105 removed from the position they would have retained within sensor carrier 710 of sheath 704, when cap 708 is in place.

Referring to FIG. 4D-G for purpose of illustration and not limitation, applicator device 20150 can be provided to a user as a single integrated assembly. FIGS. 4D and 4E provide perspective top and bottom views, respectively, of the applicator device 20150, FIG. 4F provides an exploded view of applicator device 20150 and FIG. 4G provides a side cut-away view. The perspective views illustrate how applicator device 20150 is shipped to and received by a user. The exploded and cut-away views illustrate the components of applicator device 20150. The applicator device 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only housing 20702, cap 20708, tamper evidence feature 20712, and label 20709 are visible. The tamper evidence feature 20712 can be, for example, a sticker coupled to each of housing 20702 and cap 20708, and tamper evidence feature 20712 can be damaged, for example, irreparably, by uncoupling housing 20702 and cap 20708, thereby indicating to a user that housing 20702 and cap 20708 have been previously uncoupled. These features are described in greater detail below.

### Exemplary Sensor States and Activation

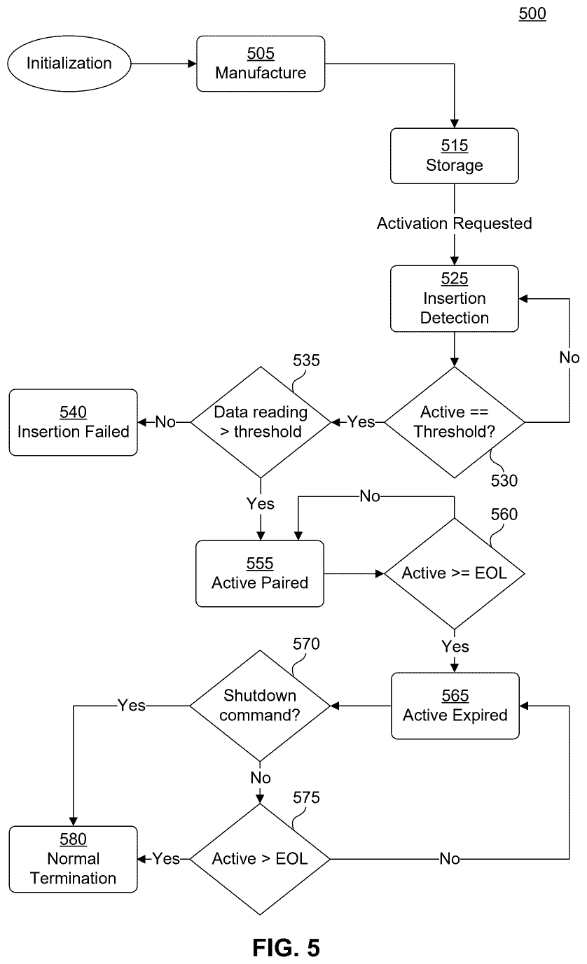

For purpose of illustration and not limitation, reference is made to the exemplary embodiment of a high-level depiction of a state machine representation 500 of the actions that can be taken by analyte sensor 110 as shown in FIG. 5. After initialization, the sensor enters state 505, which relates to the manufacture of analyte sensor 110. In the manufacture state 505, analyte sensor 110 can be configured for operation, for example, the storage memory 5030 can be written. At various times while in state 505, analyte sensor 110 checks for a received command to go to storage state 515. Upon entry to storage state 515, the sensor performs a software integrity check. While in storage state 515, the sensor can also receive an activation request command before advancing to insertion detection state 525.

Upon entry to state 525, analyte sensor 110 can store information relating to devices authenticated to communicate with the sensor as set during activation or initialize algorithms related to conducting and interpreting measurements from sensing hardware 5060. Analyte sensor 110 can also initialize a lifecycle timer, responsible for maintaining an active count of the time of operation of analyte sensor 110 and begin communication with authenticated devices to transmit recorded data. While in insertion detection state 525, the sensor can enter state 530, where analyte sensor 110 checks whether the time of operation is equal to a predetermined threshold. This time of operation threshold can correspond to a timeout function for determining whether an insertion has been successful. If the time of operation has reached the threshold, analyte sensor 110 advances to state 535, in which analyte sensor 110 checks whether the average data reading is greater than a threshold amount corresponding to an expected data reading volume for triggering detection of a successful insertion. If the data reading volume is lower than the threshold while in state 535, the sensor advances to state 540, corresponding to a failed insertion. If the data reading volume satisfies the threshold, the sensor advances to active paired state 555.

Active paired state 555 of analyte sensor 110 reflects the state while analyte sensor 110 is operating as normal by recording measurements, processing the measurements, and reporting them as appropriate. While in active paired state 555, analyte sensor 110 sends measurement results or attempts to establish a connection with data receiving device 120. Analyte sensor 110 also increments the time of operation. Once analyte sensor 110 reaches a predetermined threshold time of operation (e.g., once the time of operation reaches a predetermined threshold), analyte sensor 110 transitions to active expired state 565. Active expired state 565 of analyte sensor 110 reflects the state while analyte sensor 110 has operated for its maximum predetermined amount of time.

While in active expired state 565, analyte sensor 110 can generally perform operations relating to winding down operation and ensuring that the collected measurements have been securely transmitted to receiving devices as needed. For example, while in active expired state 565, analyte sensor 110 can transmit collected data and, if no connection is available, can increase efforts to discover authenticated devices nearby and establish and connection therewith. While in active expired state 565, analyte sensor 110 can receive a shutdown command at state 570. If no shutdown command is received, analyte sensor 110 can also, at state 575, check if the time of operation has exceeded a final operation threshold. The final operation threshold can be based on the battery life of analyte sensor 110. The normal termination state 580 corresponds to the final operations of analyte sensor 110 and ultimately shutting down analyte sensor 110.

Before a sensor is activated, ASIC 5000 resides in a low power storage mode state. The activation process can begin, for example, when an incoming RF field (e.g., NFC field) drives the voltage of the power supply to the ASIC 5000 above a reset threshold, which causes analyte sensor 110 to enter a wake-up state. While in the wake-up state, ASIC 5000 enters an activation sequence state. ASIC 5000 then wakes communication subsystem 5040. Communication subsystem 5040 is initialized, triggering a power on self-test. The power on self-test can include ASIC 5000 communicating with communication subsystem 5040 using a prescribed sequence of reading and writing data to verify the memory and one-time programmable memory are not corrupted.

When ASIC 5000 enters the measurement mode for the first time, an insertion detection sequence is performed to verify that analyte sensor 110 has been properly installed onto the patient's body before a proper measurement can take place. First, analyte sensor 110 interprets a command to activate the measurement configuration process, causing the ASIC 5000 to enter measurement command mode. Analyte sensor 110 then temporarily enters the measurement lifecycle state to run a number of consecutive measurements to test whether the insertion has been successful. Communication subsystem 5040 or ASIC 5000 evaluates the measurement results to determine insertion success. When insertion is deemed successful, analyte sensor 110 enters a measurement state, in which analyte sensor 110 begins taking regular measurements using sensing hardware 5060. If analyte sensor 110 determines that the insertion was not successful, analyte sensor 110 is triggered into an insertion failure mode, in which ASIC 5000 is commanded back to storage mode while communication subsystem 5040 disables itself.

### Exemplary Over-the-Air Updates

FIG. 1B further illustrates an example operating environment for providing over-the-air (“OTA”) updates for use with the techniques described herein. An operator of analyte monitoring system 100 can bundle updates for data receiving device 120 or analyte sensor 110 into updates for an application executing on multi-purpose data receiving device 130. Using available communication channels between data receiving device 120, multi-purpose data receiving device 130, and analyte sensor 110, multi-purpose data receiving device 130 can receive regular updates for data receiving device 120 or analyte sensor 110 and initiate installation of the updates on data receiving device 120 or analyte sensor 110. Multi-purpose data receiving device 130 acts as an installation or update platform for data receiving device 120 or analyte sensor 110 because the application that enables the multi-purpose data receiving device 130 to communicate with analyte sensor 110, data receiving device 120 and/or remote application server 155 can update software or firmware on data receiving device 120 or analyte sensor 110 without wide-area networking capabilities.

As embodied herein, remote application server 155 operated by the manufacturer of analyte sensor 110 and/or the operator of analyte monitoring system 100 can provide software and firmware updates to the devices of analyte monitoring system 100. In particular embodiments, remote application server 155 can provides the updated software and firmware to user device 140 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 110, data receiving device 120 or analyte sensor 110 and multi-purpose data receiving device 130 establish a connection. Multi-purpose data receiving device 130 determines that a firmware or software update is available for data receiving device 120 or analyte sensor 110. Multi-purpose data receiving device 130 can prepare the software or firmware update for delivery to data receiving device 120 or analyte sensor 110. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 110. Multi-purpose data receiving device 130 can also send a command to data receiving device 120 or analyte sensor 110 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 110 receives the data for the update and the command to initiate the update from multi-purpose data receiving device 130. Data receiving device 120 can then install the firmware or software update. To install the update, data receiving device 120 or analyte sensor 110 can place or restart itself in a so-called “safe” mode with limited operational capabilities. Once the update is completed, data receiving device 120 or analyte sensor 110 re-enters or resets into a standard operational mode. Data receiving device 120 or analyte sensor 110 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 5030 of analyte sensor 110 includes OTP memory. The term OTP memory can refer to memory that includes access restrictions and security to facilitate writing to particular addresses or segments in the memory a predetermined number of times. Memory 5030 can be prearranged into multiple pre-allocated memory blocks or containers. The containers are pre-allocated into a fixed size. If storage memory 5030 is OTP memory, the containers can be considered to be in a non-programmable state. Additional containers which have not yet been written to can be placed into a programmable or writable state. Containerizing storage memory 5030 in this fashion can improve the transportability of code and data to be written to storage memory 5030. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

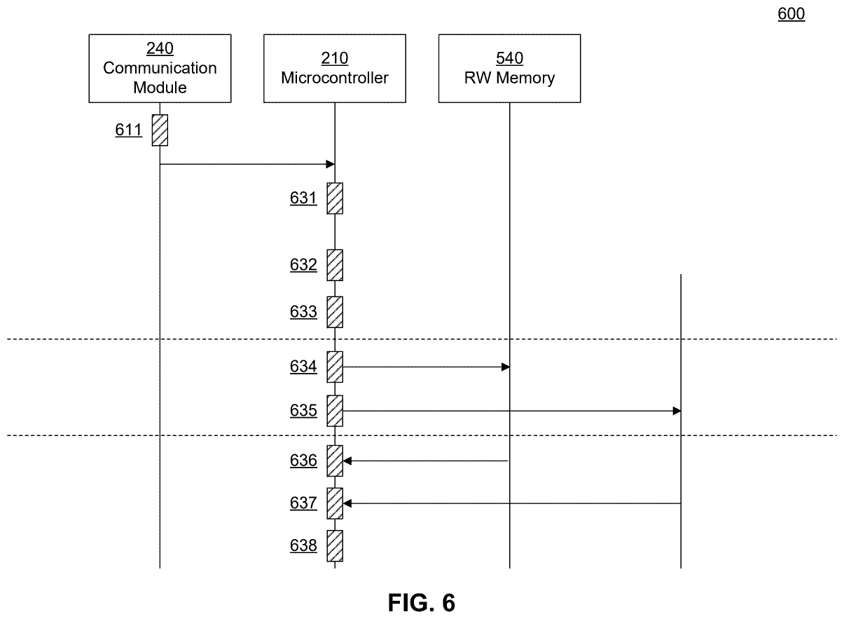

FIG. 6 is a diagram illustrating an example operational and data flow for over-the-air (“OTA”) programming of storage memory 5030 in analyte monitoring system 100 as well as use of the memory after the OTA programming in execution of processes by analyte sensor 110 according to the disclosed subject matter. In the example OTA programming 600 illustrated in FIG. 6, a request is sent from an external device (e.g., multi-purpose data receiving device 130) to initiate OTA programming (or re-programming). At 611, communication subsystem 240 of analyte sensor 110 receives an OTA programming command. Communication subsystem 240 sends the OTA programming command to the microcontroller 210 of analyte sensor 110.

At 631, after receiving the OTA programming command, microcontroller 210 validates the OTA programming command. Microcontroller 210 can determine, for example, whether the OTA programming command is signed with an appropriate digital signature token. Upon determining that the OTA programming command is valid, microcontroller 210 can set the sensor device into an OTA programming mode. At 632, microcontroller 210 can validate the OTA programming data. At 633, microcontroller 210 can reset analyte sensor 110 to re-initialize analyte sensor 110 in a programming state. Once analyte sensor 110 has transitioned into the OTA programming state, microcontroller 210 can begin to write data to the rewriteable memory (e.g., memory 5020) of the sensor device at 634 and write data to OTP memory 550 of the sensor device at 635 (e.g., storage memory 5030). The data written by the microcontroller 210 can be based on the validated OTA programming data. Microcontroller 210 can write data to cause one or more programming blocks or regions of OTP memory 550 to be marked invalid or inaccessible. The data written to the free or unused portion of OTP memory 550 can be used to replace invalidated or inaccessible programming blocks of OTP memory 550. After microcontroller 210 writes the data to the respective memories at 634 and 635, microcontroller 210 can perform one or more software integrity checks to ensure that errors were not introduced into the programming blocks during the writing process. Once microcontroller 210 is able to determine that the data has been written without errors, microcontroller 210 can resume standard operations of the sensor device.

In execution mode, at 636, microcontroller 210 can retrieve a programming manifest or profile from the rewriteable memory. The programming manifest or profile can include a listing of the valid software programming blocks and can include a guide to program execution for analyte sensor 110. By following the programming manifest or profile, microcontroller 210 can determine which memory blocks of OTP memory 550 are appropriate to execute and avoid execution of out-of-date or invalidated programming blocks or reference to out-of-date data. At 637, microcontroller 210 can selectively retrieve memory blocks from OTP memory 550. At 638, microcontroller 210 can use the retrieved memory blocks, by executing programming code stored or using variable stored in the memory.

### Exemplary Security and Other Architecture Features

As embodied herein a first layer of security for communications between analyte sensor 110 and other devices can be established based on security protocols specified by and integrated in the communication protocols used for the communication. Another layer of security can be based on communication protocols that necessitate close proximity of communicating devices. Furthermore certain packets and/or certain data included within packets can be encrypted while other packets and/or data within packets is otherwise encrypted or not encrypted. Additionally or alternatively, application layer encryption can be used with one or more block ciphers or stream ciphers to establish mutual authentication and communication encryption with other devices in the analyte monitoring system 100.

ASIC 5000 of analyte sensor 110 can be configured to dynamically generate authentication and encryption keys using data retained within storage memory 5030. Storage memory 5030 can also be pre-programmed with a set of valid authentication and encryption keys to use with particular classes of devices. ASIC 5000 can be further configured to perform authentication procedures with other devices using received data and apply the generated key to sensitive data prior to transmitting the sensitive data. The generated key can be unique to analyte sensor 110, unique to a pair of devices, unique to a communication session between analyte sensor 110 and other device, unique to a message sent during a communication session, or unique to a block of data contained within a message.

Both analyte sensor 110 and data receiving device 120 can ensure the authorization of the other party in a communication session to, for example, issue a command or receive data. In particular embodiments, identity authentication can be performed through two features. First, the party asserting its identity provides a validated certificate signed by the manufacturer of the device or the operator of analyte monitoring system 100. Second, authentication can be enforced through the use of public keys and private keys, and shared secrets derived therefrom, established by the devices of analyte monitoring system 100 or established by the operator of the analyte monitoring system 100. To confirm the identity of the other party, the party can provide proof that the party has control of its private key.

The manufacturer of analyte sensor 110, data receiving device 120, or provider of the application for multi-purpose data receiving device 130 can provide information and programming necessary for the devices to securely communicate through secured programming and updates. For example, the manufacturer can provide information that can be used to generate encryption keys for each device, including secured root keys for analyte sensor 110 and optionally for data receiving device 120 that can be used in combination with device-specific information and operational data (e.g., entropy-based random values) to generate encryption values unique to the device, session, or data transmission as need.

Analyte data associated with a user is sensitive data at least in part because this information can be used for a variety of purposes, including for health monitoring and medication dosing decisions. In addition to user data, analyte monitoring system 100 can enforce security hardening against efforts by outside parties to reverse-engineering. Communication connections can be encrypted using a device-unique or session-unique encryption key. Encrypted communications or unencrypted communications between any two devices can be verified with transmission integrity checks built into the communications. Analyte sensor 110 operations can be protected from tampering by restricting access to read and write functions to the memory 5020 via a communication interface. The sensor can be configured to grant access only to known or “trusted” devices, provided in a “whitelist” or only to devices that can provide a predetermined code associated with the manufacturer or an otherwise authenticated user. A whitelist can represent an exclusive range, meaning that no connection identifiers besides those included in the whitelist will be used, or a preferred range, in which the whitelist is searched first, but other devices can still be used. Analyte sensor 110 can further deny and shut down connection requests if the requestor cannot complete a login procedure over a communication interface within a predetermined period of time (e.g., within four seconds). These characteristics safeguard against specific denial of service attacks, and in particular against denial of service attacks on a BLE interface.

As embodied herein, analyte monitoring system 100 can employ periodic key rotation to further reduce the likelihood of key compromise and exploitation. A key rotation strategy employed by analyte monitoring system 100 can be designed to support backward compatibility of field-deployed or distributed devices. As an example, analyte monitoring system 100 can employ keys for downstream devices (e.g., devices that are in the field or cannot be feasibly provided updates) that are designed to be compatible with multiple generations of keys used by upstream devices.

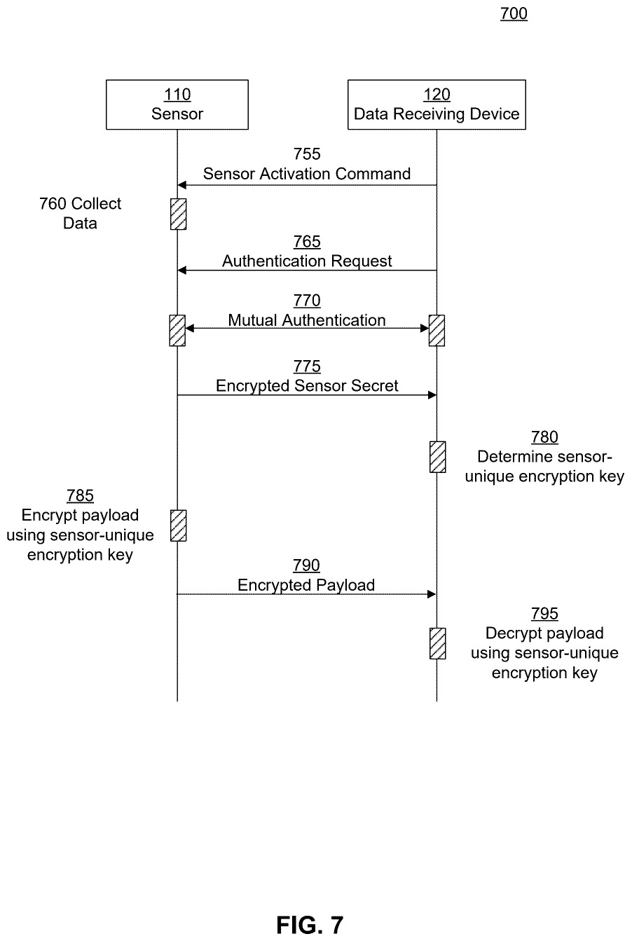

For purpose of illustration and not limitation, reference is made to the exemplary embodiment of a message sequence diagram for use with the disclosed subject matter as shown in FIG. 7 and demonstrating an example exchange of data between a pair of devices, particularly analyte sensor 110 and data receiving device 120. Data receiving device 120 can, as embodied herein, be data receiving device 120 or multi-purpose data receiving device 130. At step 755, data receiving device 120 can transmit a sensor activation command to analyte sensor 110, for example via a short-range communication protocol. Analyte sensor 110 can, prior to step 755 be in a primarily dormant state, preserving its battery until full activation is needed. After activation during step 760, analyte sensor 110 can collect data or perform other operations as appropriate to the sensing hardware 5060 of analyte sensor 110. At step 765, data receiving device 120 can an initiate authentication request command. In response to the authentication request command, both analyte sensor 110 and data receiving device 120 can engage in a mutual authentication process in step 770. Step 770 can involve the transfer of data, including challenge parameters that allow analyte sensor 110 and data receiving device 120 to ensure that the other device is sufficiently capable of adhering to an agreed-upon security framework described herein. Mutual authentication can be based on mechanisms for authentication of two or more entities to each other with or without on-line trusted third parties to verify establishment of a secret key via challenge-response. Mutual authentication can be performed using two-, three-, four-, or five-pass authentication, or similar versions thereof.

Following a successful step 770, at step 775 analyte sensor 110 can provide data receiving device 120 with a sensor secret. The sensor secret can contain sensor-unique values and be derived from random values generated during manufacture. The sensor secret can be encrypted prior to or during transmission to prevent third-parties from accessing the secret. The sensor secret can be encrypted via one or more of the keys generated by or in response to the mutual authentication process. At step 780, data receiving device 120 can derive a sensor-unique encryption key from the sensor secret. The sensor-unique encryption key can further be session-unique. As such, the sensor-unique encryption key can be determined by each device without being transmitted between analyte sensor 110 or data receiving device 120. At step 785, analyte sensor 110 can encrypt data to be included in payload. At step 790, analyte sensor 110 can transmit the encrypted payload 740 to data receiving device 120 using the communication link established between the appropriate communication models of analyte sensor 110 and data receiving device 120. At step 795, data receiving device 120 can decrypt the payload using the sensor-unique encryption key derived during step 780. Following step 795, analyte sensor 110 can deliver additional (including newly collected) data and data receiving device 120 can process the received data appropriately.

As discussed herein, analyte sensor 110 can be a device with restricted processing power, battery supply, and storage. The encryption techniques used by analyte sensor 110 (e.g., the cipher algorithm or the choice of implementation of the algorithm) can be selected based at least in part on these restrictions. Data receiving device 120 can be a more powerful device with fewer restrictions of this nature. Therefore, data receiving device 120 can employ more sophisticated, computationally intense encryption techniques, such as cipher algorithms and implementations.

### Using Model(s) for Overnight Hypo Risk

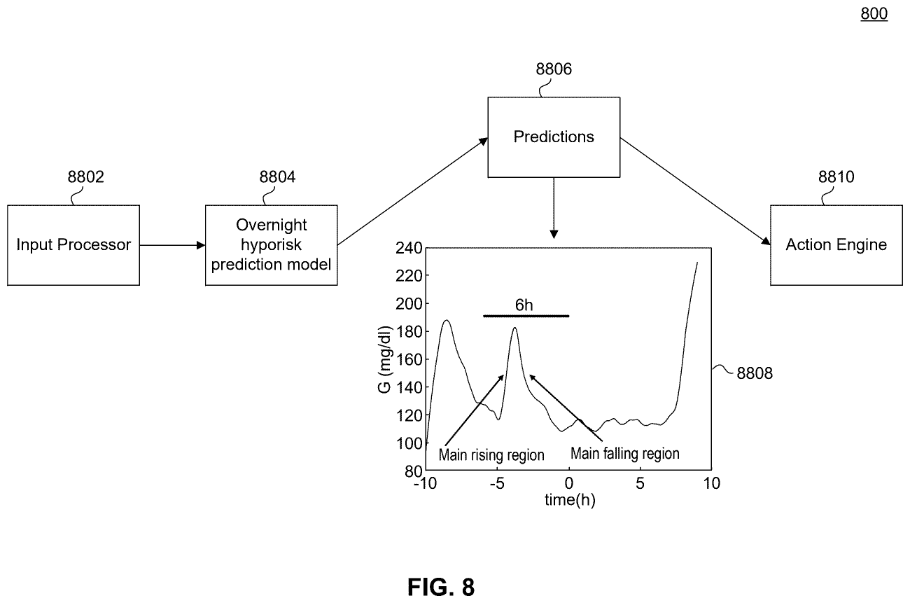

Embodiments are directed to using one or more prediction models, optionally one or more machine learning prediction models, for driving the prediction of overnight hypo risk. FIG. 8 depicts a system 800 for implementing a prediction model(s), including a machine learning prediction model(s) for generating overnight hypo risk predictions and updating potential actions in response to hypo risk predictions. System 800 can be implemented by a mobile device and/or a computing device. For example, system 800 can be implemented in one or more of data receiving device 120, multi-purpose data receiving device 130, user device 140, remote application server 155, local computer system 170, trusted computer system 180, or the like. System 800 can be implemented by one or more of processor 222 of data receiving device 120, processor 166 of analyte sensor 110, a processor of remote application server 155, a processor of user device 140, a processor of multi-purpose user receiving device 130, a processor of local computer system 170, a processor of trusted computer system 180, and any other processor.

Input processor 8802 of system 800 can provide input data to an overnight hypo risk prediction model 8804, which can be used to drive predictions 8806 based on past and/or current data and can additionally be used to (further) train prediction models. Input processor 8802 can be configured to perform preprocessing of input data prior to submission to overnight hypo risk prediction model 8804 in order to format the input data for use by overnight hypo risk prediction model 8804. Suitable input data format depends on the data that the overnight hypo risk prediction model 8804 is trained to use, as discussed further herein. In general, the format of the training data will correspond to the input data (once any preprocessing has been performed).

In some embodiments, overnight hypo risk prediction model 8804 is implemented in a user device, such as one or more of data receiving device 120, multi-purpose data receiving device 130, user device 140. In some embodiments, overnight hypo risk prediction model 8804 is implemented remote from user devices, such as at a server like remote application server 155 or local computer system 170. In some embodiments, different components of overnight hypo risk prediction model 8804 may be distributed between user devices and servers.

In some embodiments, overnight hypo risk prediction model 8804 refers to a standard machine learning model in combination with a one or more modular software components for processing inputs to overnight hypo risk prediction model 8804 and processing outputs from overnight hypo risk prediction model 8804. For example, overnight hypo risk prediction model 8804 can refer to a machine learning model operating with a input processing component for providing customized prompts and glucose data to the model. As another example, overnight hypo risk prediction model 8804 can also refer to a machine learning model operating with an output processing component for receiving outputs (e.g., an overnight hypo risk prediction) from overnight hypo risk prediction model 8804 and processing them based on rules or conditions for generating the recommendations or actions based on the prediction.

Overnight hypo risk prediction model 8804 can be implemented using one or more machine learning models to address a primary task of generating extended time range glycemic predictions and recommendations based on the predictions. In some embodiments, overnight hypo risk prediction model 8804 is configured to operate in multiple phases, including an evaluation phase, a starting phase, and a personalization phase.

In an evaluation phase, overnight hypo risk prediction model 8804 can be trained to evaluate the overnight hypo risk prediction whether to activate or recommend the overnight hypo risk prediction feature for the user. In some embodiments, overnight hypo risk prediction model 8804 evaluates the overnight hypo risk prediction in combination with continuous glucose monitoring (CGM) data of the user representing the user's glucose data for a particular time period (e.g., 2 days, 1 week, 2 weeks). The CGM data may be collected over the particular time period, can be historic CGM data that has been previously collected for the particular time period, or a combination of new and historic CGM data that collectively represent the user's data for the particular time period.

In some embodiments, overnight hypo risk prediction model 8804 is configured to automatically activate the overnight hypo risk prediction feature based on the overnight hypo risk prediction. For example, overnight hypo risk prediction model 8804 may perform a threshold comparison of the overnight hypo risk prediction and activate the prediction feature if the prediction is above a particular threshold. The prediction can be a percentage representing a likelihood of overnight hypo risk.

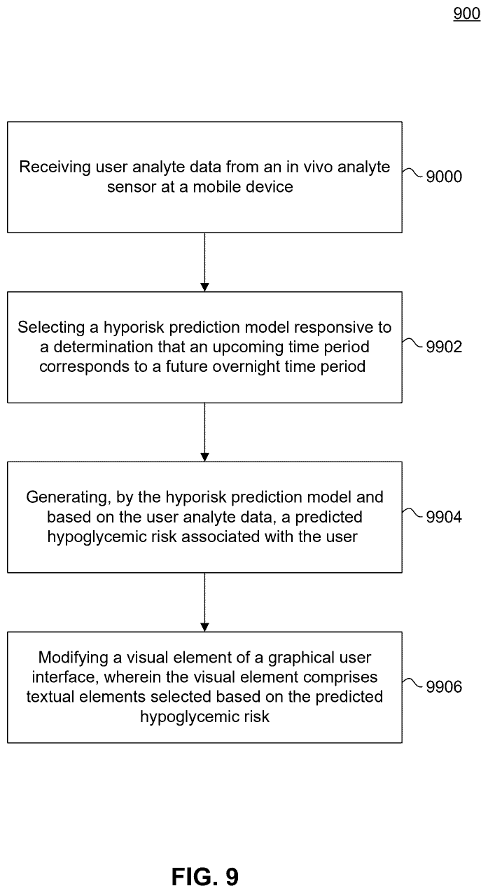

In some embodiments, overnight hypo risk prediction model 8804 is configured to generate a recommendation for the user to activate the overnight hypo risk predication feature, such as via a modification of a graphical user interface (discussed in further detail with respect to FIG. 9). For example, overnight hypo risk prediction model 8804 can be configured to display a setting on the graphical user interface for receiving user input for manually activating the prediction feature.

Evaluation of the CGM data for the user results determining whether the user will benefit from activating the overnight hypo risk prediction feature. Examples of factors for the evaluation include if the user is prone to overnight hypo (e.g., a pattern of overnight hypoglycemia over a predetermined time period, an average number of overnight low glucose alarms for a predetermined time period such as a few days, one week, or more) and has a consistent daily schedule (e.g., the overnight hypo risk prediction feature may not be beneficial for if sleep periods vary drastically from day to day by hours). A pattern of overnight hypoglycemia may be determined based on an average number of overnight low glucose alarms for a particular time period. One example threshold range is between 0.5 and 4 overnight low glucose alarms per week. In some embodiments, the threshold range for overnight low glucose alarms is the same for all users. In some embodiments, the threshold range may be adjusted on a per user basis such that users may be associated with different ranges for their overnight alarms. In some embodiments, overnight hypo risk prediction model 8804 is configured to receive information for overnight low glucose alarms from patients with diabetes (PWD) and/or caregivers of PWDs.

The starting phase can be initiated subsequent to the evaluation phase after a user is identified in the evaluation phase as a candidate for overnight hypo risk prediction. In the starting phase, in some embodiments, an initial overnight hypo risk prediction model can be utilized. In some embodiments, the initial overnight hypo risk prediction model is a pre-trained model based on initial population training data. The initial overnight hypo risk prediction model can be utilized for users that have no or insufficient glucose information for updating the prediction model. In some embodiments, for users that have sufficient glucose information (e.g., historical glucose data over a predetermined time period, glycemic patterns over a predetermined time period, average number of overnight low glucose alerts for a predetermined time period), then the initial overnight hypo risk prediction model may be updated or further trained based on the glucose information to begin personalizing the initial overnight hypo risk prediction model. This step may be performed for users having no or insufficient glucose information once the users have sufficient glucose information.

The personalization phase is initiated after the starting phase when there is sufficient glucose information about the user. The result of the personalization phase is overnight hypo risk prediction model 8804, which is generated based on continued updating of the initial overnight hypo risk prediction model and represents a personalized version of the initial (i.e., pre-trained) overnight hypo risk prediction model.

In some embodiments, overnight hypo risk prediction model 8804 is generated after sufficient glucose information is stored and accessible to the initial overnight hypo risk prediction model. Glucose information can be considered sufficient for updating overnight hypo risk prediction model 8804 based on glucose information collected over a threshold period of time. Examples of glucose information include monitored glucose data, number of overnight low glucose alarms, glycemic patterns such as a threshold number of times that glucose levels exceeded a threshold amount or were below a threshold amount. Examples of threshold period of time is one week, two weeks, or three weeks or longer. The threshold period of time may be configured to initial predetermined amount and can be adjusted upwards or downwards as part of the personalization phase so that the threshold amounts can be specific to each user.

When sufficient glucose information is collected, overnight hypo risk prediction model 8804 can be generated (and continuously updated as more glucose information is collected) from the initial overnight hypo risk prediction model. The initial overnight hypo risk prediction model is re-trained based on the glucose information which personalize the output of the overnight hypo risk prediction model 8804 such that different outputs are personalized to each user, even if the overnight hypo risk prediction model 8804 receives the same inputs for each user.

In some embodiments, overnight hypo risk prediction model 8804 can be installed as a component of a software application installed on a user device (e.g., data receiving device 120 and/or multi-purpose data receiving device 130). In such embodiments, training of overnight hypo risk prediction model 8804 can be initiated based on a personalized probability threshold, which can be generated based on the user's glucose information. Training and re-training of overnight hypo risk prediction model 8804 is performed as glucose information is collected. In some embodiments, re-training of overnight hypo risk prediction model 8804 may occur on a predetermined schedule (e.g., every week, every two weeks, every month), based on an event trigger (e.g., when a sufficient amount of additional glucose information has been collected), and/or based on user input (e.g., user initiation or request). In such embodiments, overnight hypo risk prediction model 8804 is updated as needed and does not require full-scale model re-training.

In some embodiments, overnight hypo risk prediction model 8804 can be implemented on a cloud environment. In such embodiments, overnight hypo risk prediction model 8804 can be trained based on population or sub-population data which provides population-based personalization of overnight hypo risk prediction model 8804. Such personalization provides partial re-training of overnight hypo risk prediction model 8804 for a particular sub-population by aggregating data from a cohort of users sharing a common trait or traits. Examples of traits include gender, age, and glucose patterns.

In some embodiments, functions of overnight hypo risk prediction model 8804 can be implemented in a distributed manner between a user device and a cloud environment. Examples of functions include user-specific training of overnight hypo risk prediction model 8804, generating overnight hypo risk predictions, and generating recommendations based on the overnight hypo risk predictions.

The personalization phase is a continuous and periodic process, which allows for overnight hypo risk prediction model 8804 to be updated as additional glucose information is collected. In some embodiments, the personalization phase of overnight hypo risk prediction model 8804 can include a termination condition and/or a re-start condition. The termination condition is a trigger for deactivating the overnight hypo risk prediction model 8804 for the user. The re-start condition is a trigger for restarting the evaluation phase with an initial overnight hypo risk prediction model and generating a new overnight hypo risk prediction model 8804. One or both conditions can be triggered by tracked performance of overnight hypo risk prediction model 8804 for the user. Tracking performance can include tracking accuracy of predictions and recommendations of overnight hypo risk prediction model 8804. Performance can be affected if the users change from the original user from the original evaluation phase and/or a threshold glycemic pattern change for a predetermined number of times.

As part of the personalization phase, relevant user datasets associated with the user are identified and collected for updating overnight hypo risk prediction model 8804 to generate overnight predictions that are personalized to a respective user. In some embodiments, overnight hypo risk prediction model 8804 can further be configured to generate recommendations for display on a user device corresponding to the overnight predictions. The user datasets may originate from various sources, such as stored data on the user's device, stored data by an application installed on the user device, data stored remotely such as in databases or servers. Examples of data in the datasets include but are not limited to user medical information such as glucose levels, glucose levels, historical medical data, glycemic patterns over time, and alarm history including overnight low glucose alarms.

The dataset can comprise multiple records, where each record includes one or more features. For example, for user medical information, the records can include physiological measurements such as glucose levels and glycemic patterns. Records can be numeric and text-based records obtained from users' medical or user devices (e.g., analyte sensor 110), continuous glucose monitoring applications stored on such devices, and health records. Features extracted from these records may include numerical values like average glucose level, other health data such as blood pressure range, and cholesterol readings, average number of overnight low glucose alarms, glycemic patterns for an overnight period, and user metadata such as age, weight, and health conditions.

Relevant features are selected from the dataset to personalize an initial overnight hypo risk prediction model to generate overnight hypo risk prediction model 8804 as part of the personalization phase. Feature selection techniques can be used to identify which features have the most influence for providing accurate predictions of health impact (e.g., glycemic impact) and for providing accurate detection of meal components within multi-modal data. In some aspects, a combination of filter methods, wrapper methods, and embedded methods may be used. Filter methods are useful for identifying a base dataset based on, for example, correlation analysis to identify relationships between features (e.g., carbohydrate levels) to the target variable (e.g., glycemic impact). A wrapper method is useful for refining the base dataset by ranking features based on their importance in meal component detection and glycemic impact prediction, based on, for example, recursive feature elimination for recursively eliminating redundant features such as overlapping nutrient data. Embedded methods, such as least absolute shrinkage and selection operator (LASSO) or random forest, can be useful for assessing feature importance for wide ranges of data types, such as nutrient information, physiological data. Any combination of these techniques may be utilized to ensure that meal visualization model 1306 is provided with informative and non-redundant data so that training is efficient.

In some embodiments, different versions of overnight hypo risk prediction model 8804 can be generated, with each version being trained to generate overnight hypo risk predictions for different types (e.g., based on cohort, characteristics) of patients. For example, each version of overnight hypo risk prediction model 8804 can be trained with different populations of patient data, segmented based on one or more parameters that are common to each population such as therapy, disease state (e.g., Type 1 or Type 2 diabetes mellitus), glycemic patterns or characteristics, or life-style behaviors, just to name a few examples. As noted above, overnight hypo risk prediction model 8804 can be deployed in a software application installed on the user device. Overnight hypo risk prediction model 8804 can run either in the background periodically (e.g., on a schedule) or on demand by the patient.

In some embodiments, after generating different versions (e.g., two or more) of overnight hypo risk prediction model 8804 as part of the personalization process, system 800 may select a version of overnight hypo risk prediction model 8804 for the user as part of the personalization phase. System 800 collects and processes glucose data for the user over a predetermined period of time, such as the past two weeks or a month. System 800 can apply the collected data to each version of overnight hypo risk prediction model 8804 and can select the version of overnight hypo risk prediction model 8804 that generates accurate overnight hypo risk predictions. The selection of the version of the model that is most accurate can be based on a comparison to performance metrics for overnight hypo risk predictions. For example, the performance metric can be the highest number of predicted overnight low events compensated by a false detection rate does not exceed a predetermined amount per week. Other examples of performance metrics actual hypoglycemic events, false alarms, mean absolute error (comparison of predicted and actual glucose values), missed event rate (failure to predict an actual hypoglycemia event), and false alarm rate per night.

In some embodiments, system 800 can be used to determine if the patient would benefit from having their medication doses titrated up or down rather than activating overnight hypo risk predictions. For example, if system 800 determines that the patient has an overnight low every night, rather than enable overnight hypo risk predictions, system 800 can generate a recommendation for reducing their medication. As another example, if system 800 determines that the patient never has an overnight low, system 800 can generate a recommendation for increasing medication.

Once generated, overnight hypo risk prediction model 8804 can determine when to activate overnight hypo risk predictions for the user by initiating the evaluation phase. The hypo risk predictions can be based on accessible glucose information including continuous glucose monitoring data for the user as well as other available user data including glycemic patterns and metrics of the user over a predetermined period of time, historical glucose levels, user medical conditions, and user preferences. In some embodiments, during the starting and/or personalization phases, overnight hypo risk prediction model 8804 can generate predictions based on a threshold amount of glucose information and user input for confirming certain parameters such as confirming meal choices, confirming timeframe of insulin doses (e.g., insulin dose was greater a predetermined amount of time from the generating prediction). In some embodiments, the amount of user input (e.g., button presses) is minimized to reduce the burden on the user to initiate the prediction functions. In some embodiments, overnight hypo risk prediction model 8804 can leverage other available user data in addition to glucose information and user input, such as insulin dosing data (e.g., from connected pen or user log) or meal info (e.g., from user text/image log). Other examples of user data include specific user events including events, e.g. insulin dose, meal, exercise (from fitness device), historic data (e.g., over 8 hours, 24 hours, 14 days, 28 days), and recent scan data.

During the starting and/or personalization phases, overnight hypo risk prediction model 8804 is also configured to generate recommendations based on the overnight predictions. Examples of recommendations include possible interventions, which can be tailored based on analysis of user glycemic patterns and knowledge of their existing therapy, recommending a bedtime snack if overnight hypo risk prediction is high, recommending a one-time reduction of basal dose amount if overnight hypo risk prediction is high, for users on multiple daily injections or basal only therapy, and adjusting target glucose (as a way to reduce basal infusion rate) for an automated insulin delivery user.

During the starting and/or personalization phases, overnight hypo risk prediction model 8804 is also configured to generate recommendations and other visual content based on overnight hypo risk predictions. Examples of recommendations include eating food or nutrition of a particular carbohydrate amount, adjustment to medication such as a one-time adjustment (e.g., reduction) of basal dose amount if overnight hypo risk prediction is high, and/or counseling with HCP for adjustment of insulin regimen or other medication, if overnight hypo occurs frequently, for users on insulin and/or other medication.

In some embodiments, overnight hypo risk prediction model 8804 can be a customized multimodal large language trained to generate the customized overnight hypo risk predictions for each user associated with a device on which overnight hypo risk prediction model 8804 is installed. In some embodiments, overnight hypo risk prediction model 8804 can be implemented as a pre-trained/pre-existing multimodal large language models (LLM).

In some embodiments, outputs of overnight hypo risk prediction model 8804 can be a probability or likelihood of a condition (i.e. hypoglycemic condition) occurring in the future over an extended time horizon (i.e. overnight period), or a prediction of a condition (i.e. hypoglycemic condition) occurring in the future over an extended time horizon (i.e. overnight period) based on exceeding a likelihood threshold. These outputs, i.e. predictions 8806, can be utilized in a visualization 8808 that can be displayed, for example as a visual element on a graphical user interface, or otherwise utilized by overnight hypo risk prediction model 8804.

In some embodiments, an example of an input to an overnight hypo risk prediction model 8804 includes user medical history, including continuous glucose data associated with a user over a past time-period. The past-time period can end at the current time. The past-time period can correspond to a predetermined period of time such, a previous 12 hour time period, 16 hour time period, 24 hour time period, 36 hour time period, or 48 hour time period, as further discussed herein. In some embodiments, an output of overnight hypo risk prediction model 8804, i.e. predictions 8806, is a likelihood of a hypoglycemic condition occurring during an upcoming time-period, in particular, an extended time-period such as a future overnight time period. The upcoming time-period can be determined to be a future overnight time period by one or more of: the time of day, such as after 10 P.M; the number of hours, such as 7 hours, 8 hours, or more; by particular user events, such as a sleep event, that can be defined by a start and end condition (e.g., going to bed, waking up); or directly via user input to the mobile device and/or computing device. A hypoglycemic condition can include a scenario where a user's glucose level falls below a predefined threshold such as 70 or 54 mg/dL.

In some embodiments, as described further herein, multiple prediction models could be implemented, for instance, each with a different threshold level for the hypoglycemic condition, and the model that is enabled can be configured by or for the user.

In some embodiments, as further described herein, overnight hypo risk prediction model 8804 can also have an output that is a combination of multiple condition likelihoods and conditions. For instance, a threshold can be defined by a continuum where different thresholds can be established for different glucose levels (e.g., at 70 mg/dL the likelihood must be 90% or more, but at 54 mg/dL the likelihood must be 50% or more).

System 800 can also be implemented with an action engine 8810 which is configured to operate in combination with overnight hypo risk prediction model 8804 durin both the starting and/or personalization phases to perform actions based on the overnight hypoglycemia prediction. Action engine 8810 can further be implemented as a machine learning model trained to generate alerts and textual recommendations based on analysis of the overnight hypoglycemia prediction. Action engine 8810 can be further trained on population data, cohort data, personalized user data, or a combination of both, to provide personalized actions that a user is more likely to take. Examples of actions include generating alerts, generating recommendations, personalizing the visualization 8808 with user-specific tasks or action items to take to mitigate the overnight hypoglycemia prediction. Recommendations can include predefined textual actions that can be selected by action engine 8810 based on a combination of the overnight hypoglycemia prediction and user data.

Personalizing the visualization 8808 can include displaying the overnight hypoglycemia prediction on a blood glucose graph as an extension of a user's actual glucose levels (e.g., a visual indication that the user will likely experience overnight hypoglycemia). The overnight hypoglycemia prediction can be displayed using a visual identifier that is distinct from a visual identifier for the user's actual glucose level such that a viewer can visibly distinguish between the overnight hypoglycemia prediction and the actual glucose levels. In some embodiments, personalization the visualization 8808 further include depicting more than one overnight hypoglycemia prediction that can be tuned according to various user provided inputs, such as a potential meal (e.g., low carbohydrate meal) or activity (e.g., strenuous exercise). For example, visualization 8808 can display an overnight hypoglycemia prediction for a selected meal (or a skipped meal) and an overnight hypoglycemia prediction for a selected activity.

In some embodiments, action engine 8810 is configured to generate a personalized visual indication of the overnight hypo risk prediction for display on a personalized graphical user interface (further discussed with respect to FIG. 9) As one example, action engine 8810 converts output from overnight hypo risk prediction model 8804 into a quantifiable visualization such as a number (e.g., for a particular range such as from 1—very low hypo risk to 10—significant hypo risk), a percentage (e.g., representing a likelihood of hypo risk such as between 0% and 100%), risk level indication (e.g., low, medium, high), color coded visualizations such as a green, yellow, and red icon corresponding to the level (e.g., low, medium, and high) of predicted hypo risk.

Action engine 8810 can also store historical user data such as past interaction data and past action data that provides information about how users have previously interacted with visualization 8808 and how users have previously responded to past recommendations or alerts. For example, action engine 8810 can determine that certain visual elements are more effective (i.e., received higher rates of interaction) and select those visual elements for use in visualization 8808. As another example, action engine 8810 can determine that certain types of alerts or notifications received higher rates of action from a user and can select those types of alerts or notifications when generating the alerts and recommendations.

### Exemplary Models and Training

Prior to the evaluation, starting, and personalization phases, overnight hypo risk prediction model 8804 can be trained or otherwise refined using different implementations of model development: 1) feature engineering-based or 2) deep-learning based. In a feature engineering-based approach, a collection of numerical values called features can be extracted from a collection of glucose data (e.g., for a specific user, multiple users, or a cohort of users) in order to train or otherwise refine the models and to use the model for the overnight prediction. Models can be trained or otherwise refined for a specific user, for multiple users, or for a cohort of users depending on the glucose data (or other CGM data) that is used for training. Training or refining for a specific user is referred to herein as a personalized prediction model, whilst training for a cohort of users is referred to as a population prediction model. Population prediction models can be transformed into personalized prediction models, as discussed further herein, or personalized prediction models can be individually trained.

The feature extraction methods are specifically developed for extracting the information most relevant to an overnight hypoglycemia prediction. For example, feature-based approaches can be based on an integration of domain knowledge and machine learning approaches, which is in contrast to deep learning approaches that automate the feature extraction process and take in raw glucose data for the deep learning based model training.

When utilizing feature engineering-based model development for the overnight hypo risk prediction model, this disclosure describes advantages of this feature-engineering based approach including a) easier to utilize the domain knowledge since feature-based approaches can be based on an integration of domain knowledge and machine learning approaches, b) building of a smaller model, and c) the model is more interpretable allowing outputs provided by the overnight prediction model and the reasoning for said outputs to be better understood. A smaller model is beneficial because smaller models can be deployed to the edges of glucose monitoring systems (i.e. system 100), such as mobile devices (e.g. data receiving device 120 and/or multi-purpose data receiving device 130), rather than centrally, such as in a server (e.g. at a cloud-based system such as trusted computer system 180). Advantages of using deep-learning based models are discussed elsewhere herein.

Development of the hypo risk prediction model includes multiple steps including obtaining and pre-processing glucose data, feature engineering and feature extraction, model training, and model evaluation. Details of these steps are described below.

Obtaining training data. In some embodiments, training of the hypo risk prediction model(s) can include data collection and labeling steps. The training data includes at least time-series glucose data (also referred to herein as a glucose trace). The training data can optionally include other CGM data and other patient data. Training data can be real-world historic data, synthetic data, or a combination thereof.

In some embodiments, the data collection step can involve retrieving historic real-world glucose data for one or more cohorts of users (e.g. 1,000 users) of a user age type (e.g. adult, teenagers, children) and conditions (e.g. diabetes patients, healthy subjects). Additionally or alternatively, the data collection step can involve retrieving real-world glucose data for the user specifically. Historic real-world CGM data can be collected from CGM devices (e.g. analyte sensor 110) attached to patients and collected via a cloud-based system (e.g. trusted computer system 180 and/or remote application server 155). Due to the prevalence of glucose monitoring systems (e.g. system 100), for diabetes patients as well as healthy subjects, such historic glucose data is readily available at remote application server 155 and/or trusted computer system 180. CGM data can be gathered from a diverse cohort of individuals.

In some embodiments, training outputs for training the overnight prediction model can also be retrieved. Training outputs are the actual historic measured hypoglycemic condition. Other CGM data and/or patient data that can be used to drive or train the overnight prediction model include insulin dose delivery records, other medication delivery records, patient demographic and medical information, food consumption, activity, location, events, illness, day-of-the-week, time-of-day, and calendar day (e.g., holidays).

In some embodiments, a data collection step can include simulating glucose data for one or more cohort of users (e.g., 1,000 users) of a virtual user age type (e.g., adults, teenagers, children) and conditions (e.g., type 1 diabetes patients, type 2 diabetes patients, prediabetes patients, and healthy subjects) for training overnight hypoglycemia prediction model. Additionally or alternatively, the data collection step can involve simulating glucose data for the user specifically. Such synthetic data can be generated to simulate possible scenarios not sufficiently covered by real-world data, such as overnight hypoglycemic events, or to increase the amount of training data. This can be accomplished using statistical models that replicate the underlying distribution of real-world data or through generative models like Generative Adversarial Networks (GANs). In some embodiments, simulating glucose data can comprise obtaining historic glucose data, and performing data augmentation with the historic glucose data to artificially generate further glucose data. In some embodiments, a separate model can be used for generating simulation parameters such as the number of cohorts, the particular time period, the type, and the conditions.

Glucose data takes the form of a series of raw (or simulated) measurements in mg/dL, and is also referred to herein as a glucose trace. Typical measurement values are between 54 and 200 mg/dL, but can be lower than 54 mg/dL (i.e. hypoglycemia) and above 200 mg/dL. The series of measurements are recorded over time (i.e. a time-series), and a timestamp can be assigned to each of the measurements. For instance, the timestamp can be the time passed since the overnight period began. In some embodiments, glucose data can be recorded with a predetermined sampling interval (e.g., 1 minute, 5 minutes, 10 minutes).

In some embodiments, particularly those where a personalized model is to be trained, the glucose data relates to the user. In such embodiments, a long history of CGM data (e.g. at least 2 years) is useful. For instance, overnight hypo risk prediction model 8804 can be trained using a predetermined number of days (e.g., 4000) worth of glucose traces.

In some embodiments, including those where a population prediction is determined, the glucose data relates to one or more cohorts of users, each cohort having a particular user age type and condition. In some embodiments, the glucose data can relate to one cohort of users of a specific user age type and specific condition matching the user of the trained model. In other embodiments, the glucose data can relate to more than one cohort of users of different user age type or different conditions, in order to produce a more generalized trained hypo risk model (which can then be personalized further, as described herein). Overnight hypo risk prediction model 8804 can be trained using a predetermined number of days (e.g., 4000) worth of glucose traces from a number of different patients.

In embodiments, the glucose data can be labeled to indicate which data represents past overnight periods and/or which data corresponds to 24 hours, 36 hours or 48 hours before the start of each overnight period. The labelling can be performed automatically based on other CGM data, for example the user defined overnight period, as well as timestamps associated with the glucose data. In other embodiments, it can be determined as part of data collection which data represents past overnight periods and/or which data corresponds to 24 hours, 36 hours or 48 hours before the start of each overnight period, and only this data collected. Note that collecting the glucose data of the historic overnight periods can not always be necessary since the training outputs can be used instead to indicate whether a hypoglycemic condition was present in a particular overnight period.

The glucose data can be labeled to indicate whether hypoglycemia is present. In some embodiments, individual glucose data measurements can be labeled. In some embodiments, data labeling can be based on the glucose data during the overnight (i.e., post bedtime) period (i.e., as a whole). Such labeling can be automated based on a predefined labeling rule and/or based on the corresponding training outputs. As one non-limiting example of a labeling rule, an overnight period can be labeled based on a threshold number of glucose measurements being outside of a threshold limit. For example, an overnight period can be labeled as “positive” (i.e. for hypoglycemia) when 3 or more consecutive glucose data measurements are lower than 54 mg/dL. Other values for the thresholds are possible, such as 70 mg/dL. As one examples, values for the threshold number of glucose measurements and/or the threshold limit can be personalized for each user based on the user's glucose history and exogenous parameters such as the user's medical history, user's eating history, preferences, and HCP feedback.

In some embodiments, the user's glucose history and exogenous parameters such as the user's medical history, user's eating history, preferences, and/or HCP feedback can be fed into a machine learning model trained to identify personalized threshold values, such as the threshold number of glucose measurements and a threshold limit for the glucose value. In other embodiments, personalized threshold values, such as the threshold number of glucose measurements and a threshold limit for the glucose value can be set by a HCP. Accordingly, operations of the software application based on these personalized threshold values can be different for different users. Moreover, the use of a trained machine learning model enables the threshold values to be updated dynamically and over time based on new user data. For example, the trained machine learning model can be implanted as part of the software application installed on each user's mobile device (i.e. on data receiving device 120 or multi-purpose data receiving device 130). Each localized trained machine learning model can be configured to communicate values to a central location (e.g., via a cloud network, such as at trusted computer system 180) for storage. In other embodiments, the trained machine learning model can be implemented centrally (e.g., via a cloud network, such as at trusted computer system 180) that is in communication with the software applications installed on each user's mobile device (i.e. data receiving device 120 or multi-purpose data receiving device 130).

In some embodiments, the dataset from each user or cohort can be partitioned as a training dataset and test dataset. In other embodiments, the dataset can be partitioned into training, validation, and testing datasets. The particular partitioning can be determined based on needs for accuracy or speed. For example, a dataset can be partitioned into 70% training and 30% test which can result in a longer training period but more accurate predictions by the machine learning model. A split in the dataset ensures the model can be trained extensively while also being tuned and tested against unseen data. In some embodiments, dataset partitions can be subject based, i.e., all data from a given subject were either in the training dataset or test dataset.

Preprocessing of the training data. In general, preprocessing the training data includes handling missing data, normalizing data ranges, and aligning timestamps. Suitable techniques for performing these are known in the art. In general, any preprocessing techniques described herein for the training data can also be performed by input processor 8802 for the input data. This ensures that the overnight hypo risk prediction model 8804 is trained on the form of data that it subsequently makes predictions using.

Glucose data can undergo specific preprocessing steps. In some embodiments, glucose data can be preprocessed prior to the feature extraction to improve the accuracy of training a machine learning model. Preprocessing data can improve the formatting of data prior to training by making data smoother and more evenly spaced with a particular sampling interval (e.g., 5 minutes). Input processor 8802 can be configured to perform the preprocessing step. In some embodiments, input processor 8802 can be configured to utilize a sliding window method to perform preprocessing. Sliding window refers to sliding a time window with a certain time increment with the center of the window sliding within a particular time range. For example, input processor 8802 can slide a 60-minute time window with a 5-minute increment with the center of the window sliding in the time range starting from 30 minutes (half of the window size) after the first data point to 30 minutes before the last data point.

For each time window, input processor 8802 can collect raw input data (i.e. glucose measurements) within the respective time window and fit the collected input data. For example, input processor 8802 can utilize a quadratic function to fit the collected input data and can then assign the fitted value as preprocessed data at the center of the time window. Input processor 8802 can utilize different types of data during the data preprocessing steps for each time point with 5-minutes interval. These types of data can include smoothed glucose data, glucose values estimated by the fitted model at the end point of the time window, glucose rate of change, and/or acceleration of the glucose data.

Smoothed glucose data can be estimated by input processor 8802 such as via a fitted model using the raw input data collected. The raw input data can be collected within a particular time window (e.g., 1 hour) and can be centered at a particular time point for which the preprocessed data is computed. An example of the fitted model is a quadratic function model. Parameters of the fitted model for calculating the smoothed glucose data include a glucose value estimated by the fitted model at an end point of the time window. In an example, the time window can be a 1-hour window so the centered time point would be data at the 30 minute point of the window and the end point glucose value would be data at the 1-hour point of the window (e.g., an end point of a 1-hour window).

Other parameters can be the glucose rate of change and the glucose acceleration.

In a non-limiting example, these parameters can be used by the fitted model to generate the overnight predictions for the glucose value. For example, the smoothed glucose value for a particular centered time point can be determined based on the formula G_s(t_cur)=a*t_cur\hat{ }2+b*t_cur+c, with parameters, a, b, and c are available from the fitted model, G_s representing the smoothed glucose value and t_cur representing the centered time point.

Input processor 8802 can also determine an estimated real time glucose value G_rt via the equation G_rt(t_cur)=a*(t_cur+X min)\hat{ }2+b* (t_cur+X min)+c. example, the glucose value estimated by the fitted model can be at the end point of the time window, which is X minutes after t_cur.

Input processor 8802 can also determine a glucose rate of change and glucose acceleration. In some embodiments, glucose rate of change can be determined via the equation ROC_glu(T_cur)=2*a*T_cur+b and glucose acceleration can be determined via the equation A_glu(T_cur)=a.

Feature engineering and extraction: after preprocessing, feature engineering and extraction is performed. The techniques described herein are characterizing how the input processor 8802 processes the input data, however the same techniques are performed to the training data. This ensures that the overnight hypo risk prediction model 8804 is trained on the form of data that it subsequently uses when making predictions.

Input processor 8802 can next extract information related to the overnight prediction from the preprocessed glucose data. Input processor 8802 can identify features characterizing the glucose profiles within predefined time windows. Input processor 8802 can extract features from glucose data within various predefined time windows for predicting overnight hypoglycemia. Extracted features can include: a slope computed with linear regression from glucose data collected within certain number of time windows ahead of a sleep event (e.g., four consecutive 3-hour time windows prior to bedtime), the minimum, maximum, standard deviation, glycemic variability percentage within a predefined time window prior to a sleep event prior to the time of the overnight prediction (e.g., 3-hour time window prior to the bedtime in 4 consecutive days), the glucose value at the time of the overnight prediction, mean glucose values for a predefined number of consecutive time windows prior to the time of the overnight prediction (e.g., in 6 consecutive 1-hour time window), the total area of glucose values below a predefined value (e.g., 70 mg/dL), low blood glucose index and high blood glucose index within a predefined time window prior to the overnight prediction (e.g., 6-hour time window), mean glucose values computed from a predefined number of consecutive time windows prior to the overnight prediction (e.g., 5 consecutive 3-hour time windows), time in hyperglycemia in a predefined number of consecutive time windows prior to the overnight prediction (e.g., 3 consecutive 3-hour windows), and/or the mean of the minimum of the nocturnal glucose in one week prior to the time of the overnight prediction (or any combination thereof).

One example of extracted features are features characterizing the most recent meal induced glucose excursion (latest meal excursion) prior to the time of the overnight prediction. Predictions 8806 can include a graphical visualization 8808 of the prediction along a graphical curve plotted over a predefined time span, as shown in FIG. 8. Visualization 8808 provides an example of the smoothed glucose data (G) as the function of the elapsed time from the overnight prediction time (i.e. the start of the overnight period). The time is the elapsed time from the time of the overnight prediction in hour. Negative elapsed time means prior to the overnight prediction time. Visualization 8808 can include annotations of the main rising and falling regions.

In some embodiments, visualization 8808 can be generated having extracted features characterizing latest meal excursion by the following steps. A first step involves identifying rising and falling regions within the preprocessed glucose data. As one example, overnight hypo risk prediction model 8804 can segment preprocessed glucose data as a series of monotonically increasing (rising) and decreasing (falling) regions by local minima and local maxima. Overnight hypo risk prediction model 8804 can identify a data point in this dataset as a local minimum if it is smaller than both of its left (earlier data point) and right (later data point) data point. Similarly, overnight hypo risk prediction model 8804 can identify a data point in the smooth glucose data as a local maximum if it is larger than both of its left and right data point. When values of two or more data points are equal and the value is larger/smaller than both of the first left and right non-equal data points, then overnight hypo risk prediction model 8804 can mark the last data point as a local maximum or minimum. A rising region is a segment in the smoothed glucose data beginning at a local minimum and ending at the subsequent local maximum, while a falling region is a segment beginning at a local maximum and ending at the subsequent local minimum. The height of a rising or falling region is the difference between the end and the beginning of the region.

In some embodiments, visualization 8808 can include various visual elements that are selected based on the predicted hypoglycemic risk. The visual elements can be adapted and personalized for the particular user and/or the predicted hypoglycemic risk.

This step can further include overnight hypo risk prediction model 8804 identifying the main rising and falling region of smoothed glucose data. For example, overnight hypo risk prediction model 8804 can identify a typical meal excursion by a prominent rising region and a subsequent prominent falling region. The height of the meal excursion can vary but it is typically the most prominent rising region within a 5 to 6-hour time window after a meal start time.

There are a number of ways that overnight hypo risk prediction model 8804 can identify rising and falling regions of the latest meal excursion. For example, overnight hypo risk prediction model 8804 can identify a main rising region as the highest rising region ending within a predetermined (e.g., 6 hour) time window counting back from the time of the overnight prediction. The main falling region can be identified as being the highest (largest absolute value of the height) falling region between the end of the main rising region and the time of the overnight prediction.

There can also be corner cases in locating the main rising and falling regions. For example, overnight hypo risk prediction model 8804 can identify any rising regions that ended within the predetermined time window before the time of the overnight prediction. Overnight hypo risk prediction model 8804 can be configured to identify one of the following reasons for these corner cases. For example, there can only a single rising region within the predetermined time window, in which case overnight hypo risk prediction model 8804 can pick the rising region as the main rising region, even though that region did not end by the time of the overnight prediction. In another example, overnight hypo risk prediction model 8804 can identify a falling region followed by a rising region, in which case overnight hypo risk prediction model 8804 can pick the rising region as the main rising region, even though it did not end by the time of the overnight prediction. In another example, overnight hypo risk prediction model 8804 can identify only a single falling region in which case overnight hypo risk prediction model 8804 can pick the first rising region ended before the falling region. Overnight hypo risk prediction model 8804 can determine there are no falling regions after the main rising region. In this case, overnight hypo risk prediction model 8804 replicates the rising region as the main falling region.

Another example of extracted features are features characterizing the latest meal excursion. Overnight hypo risk prediction model 8804 uses the features extracted from the main rising and falling region to characterize the latest meal excursion. The extracted features can include the time and glucose value at the beginning and end of the regions, the duration and the height of the region, the minimum, maximum and average of the glucose rate of change, minimum and maximum of the acceleration of the glucose value, and the incremental area under the curve of the region.

Overnight hypo risk prediction model 8804 can extract different types of features from the main rising and the main following region for use in generating the overnight prediction. Examples of extracted features from the main rising region and the main falling regions include one or more parameters associated with main rising and falling regions such as an elapsed time of the beginning of the main rising/falling region, an elapsed time of the end of the main rising/falling region, values at the beginning of the main rising/falling region, values at the end of the main rising/falling region, glucose values/rates within the main rising/falling region, height and duration (and metrics associated with height and duration such as ratio) of the main rising/falling regions, and/or area under the curve of the main/falling regions (or any combination thereof).

Another example of extracted features are features characterizing the glucose profile at the time of the overnight prediction. Overnight hypo risk prediction model 8804 can compute glucose values and the glucose rate of change at the time of the overnight prediction. For example, overnight hypo risk prediction model 8804 can determine glucose values at the time of prediction, which can be computed using a linear regression of the glucose values in the past 30 minutes from the time of the overnight prediction. Overnight hypo risk prediction model 8804 can determine the estimated glucose rate of change at the time of the overnight prediction by computing the slope using linear regression of the glucose values from the end of the falling region to the time of the overnight prediction. In some embodiments, overnight hypo risk prediction model 8804 can compute a slope using a linear regression of the glucose values in the past 30 minutes from the time of the overnight prediction when the main falling region is the last region before the time of the overnight prediction.

In some embodiments, a combination of extracted features can be manually selected and provided to overnight hypo risk prediction model 8804, for example by an HCP. In one embodiment, the value at the end of the main falling region, the estimated glucose rate of change at the time of the overnight prediction, the elapsed time of the beginning of the main rising region, counting back from the time of the overnight prediction, and the minimum glucose value of the first night before the time of the overnight prediction are the selected features.

In some embodiments, overnight hypo risk prediction model 8804 can be trained using feature selection. In some embodiments, overnight hypo risk prediction model 8804 can receive a predetermined number of features using forward sequential feature selection. Selected features can be prioritized based on their contribution to the accuracy of the overnight prediction. Higher prioritized features include the value at the end of the main falling region, the estimated glucose rate of change at the time of the overnight prediction, the elapsed time of the beginning of the main rising region, counting back from the time of the overnight prediction, and/or the minimum glucose value of the first night before the time of the overnight prediction (or any combination thereof). Other relevant prioritized features can include values associated with glucose values (e.g., mean, maximum, minimum) such as the mean glucose value over a predetermined period of time, the maximum glucose rate of change in a main falling region, the minimum glucose value of a particular day prior to the time of the overnight hypo risk prediction, blood glucose values, glycemic variability percentages, minimum and maximum glucose accelerations within main rising/falling regions, mean glucose values, slopes of linear progressions, and/or parameters associated with the main rising/falling regions such as glucose acceleration, duration, height, elapsed time, incremental areas (or any combination thereof).

In the development of predictive models for glucose levels, the selection of relevant features is critical to achieving high accuracy and personalization. In some embodiments, overnight hypo risk prediction model 8804 incorporates one or more advanced machine learning algorithms designed to select from the above features. This feature selection can be dynamically tailored to each user's unique patterns and conditions, enhancing the predictive power of the model.

In some embodiments, the feature selection process of overnight hypo risk prediction model 8804 can include one or more techniques including correlation-based selection, recursive feature elimination, and machine learning-based importance evaluation to identify the most predictive features for each individual. Initially, overnight hypo risk prediction model 8804 can assess the overall importance of each feature across a general population to determine a subset of features that captures the most significant indicators of glucose fluctuations.

Subsequently, overnight hypo risk prediction model 8804 can refine this feature set for each user by integrating user-specific data. This data-driven approach allows overnight hypo risk prediction model 8804 to adapt the feature importance weights according to the individual's physiological responses and historic glucose levels. For instance, if a user's glucose rate of change is a significant predictor of future levels, it can be weighted more heavily in their personalized model. Other parameters, such as insulin sensitivity indices or carbohydrate intake, can also be selected based on their relevance to the individual's glucose control.

The criteria for overnight hypo risk prediction model 8804 selecting features include statistical significance, predictive power, and consistency across similar profiles. Overnight hypo risk prediction model 8804 can prioritize features that show a high correlation with glucose levels and provide consistent predictions across multiple datasets. Moreover, in some embodiments, overnight hypo risk prediction model 8804 can employ regularization techniques to prevent overfitting by penalizing the inclusion of irrelevant features.

In some embodiments, overnight hypo risk prediction model 8804 utilizes one or more machine learning techniques to facilitate effective feature selection. Decision trees, for example, can be used to ascertain the information gain from each feature, while regression models can help in reducing the feature space to the most relevant predictors. Gradient boosting machines further refine the feature set by sequentially correcting prediction errors, which is particularly useful in personalizing the model based on new user's glucose readings.

When using decision trees, a random forest classifier can be used for training the overnight hypo risk prediction model 8804, i.e. to make a determination about whether hypoglycemia is predicted to occur in the overnight period. Negative instances can be randomly down sampled to in such a way the ratio of positive and negative instances is 1:2. Model evaluations can be performed using the entire test dataset (without resampling). Some commonly used prediction performance metrics were computed for evaluation, including the area under curve (AUC) of receiver-operating curve (ROC), sensitivity, specificity, positive and negative predictive values, and number of false positive/negative per week.

In some embodiments, in addition to the glucose data discussed above, overnight hypo risk prediction model 8804 can also be trained and evaluated using other data from CGM data collected in real-world applications, clinical studies, and/or synthetic CGM data.

Real-world CGM data can be collected from CGM devices attached to patients and collected via a cloud-based system (e.g. trusted computer system 180 and/or remote application server 155). Real-world CGM data can be gathered from a diverse cohort of individuals. In addition to the timestamps and glucose measurements, CGM data can include potentially relevant features such as meal times, carbohydrate intake, insulin administration, physical activity, and other physiological parameters like heart rate and sleep patterns, all or some combination of which can aid understanding the glucose dynamics influenced by meals, exercise, insulin injections, and other physiological factors. Using other CGM data can reduce the amount of preprocessing that needs to be performed or amount of inference that the overnight hypo risk prediction model 8804 needs to do, resulting in more accurate predictions. As before, synthetic data can be generated to simulate possible scenarios not sufficiently covered by real-world data, such as overnight hypoglycemic events. This can accomplished using statistical models that replicate the underlying distribution of real-world data or through generative models like Generative Adversarial Networks (GANs). Both real-world and synthetic CGM data can undergo preprocessing as discussed above to ensure quality and consistency. This can include handling missing data, normalizing data ranges, and aligning timestamps. Features such as the rate of change of glucose, historical averages, and time since last meal can be calculated and included during this stage.

In some embodiments, overnight hypo risk prediction model 8804 can utilize Deep Learning (DL) based model design. DL is a category of Machine Learning (ML) techniques/models that can be used for classification and prediction tasks, including the prediction of a hypoglycemic condition during an overnight period. DL-based model design can leverage complex neural network architectures that are particularly well-suited for handling the variabilities and dynamic nature of glucose data influenced by numerous physiological factors. In the sense of overnight hypo risk prediction, deep learning models, such as Convolutional Neural Networks (CNNs) and Recurrent Neural Networks (RNNs), can identify complex patterns in sequential data. In the context of time-series CGM, a deep learning model can detect patterns across multiple time steps, capturing trends and anomalies in glucose levels that other models might miss. For example, a DL model can take in the glucose trace in the past 24 hours (or of some other duration), pass it through a sophisticated and trained Artificial Neural Network (or similar type of computational models), and produce an estimated probability of occurrence of hypoglycemic condition in the next 6 hours (or of some other prediction horizon).

A key characteristic of DL technique, as compared to feature engineering based techniques, is that it requires no specific design of features of the glucose trace, but rather relies on the design of a more generic neural network structure that can self-adapt to the detection of glucose trace features that correlate to hypoglycemic condition occurrences. In other words, deep learning models inherently perform feature learning, which is the automatic identification and utilization of relevant features from raw glucose data. This capability allows these models to have improved prediction accuracy without requiring explicit specification of features. Self-adaption can be accomplished through the training process of the DL model (e.g. the neural network), feeding into it glucose traces with known occurrence or non-occurrence of a hypoglycemic condition in the window of interest (i.e. in the overnight period), so that the DL model automatically learns of the patterns and adjust its model parameters to look for the most relevant features. The result of this training process is a DL model tailored for the overnight prediction of hypoglycemic occurrence.

In some embodiments, overnight hypo risk prediction model 8804 can be implemented as a 1-Dimensional Convolutional Neural Network (1-D CNN), which is a DL technique for applications whose input is time-series data, such as the glucose traces and other CGM data described here. 1-D CNN models are particular suited for processing time-series data because time-series data can often have local patterns or relationships between neighboring time points. 1-D CNNs can be tuned for detecting such local dependencies through convolutional filters that slide across the time axis. These filters capture short-term relationships in the data, such as trends, spikes, or patterns in time steps close to each other.

The disclosure contemplates at least one 1-D CNN model for overnight hypo risk prediction implemented as a neural network comprising one or more Convolutional Layers, one or more ReLU Activation Layers, one or more Average/Max Pooling Layers, and one or more Fully-Connected Layers.

In some embodiments, the 1-D CNN model can be trained to accept, as input, a glucose trace covering a predetermined time period (e.g., 24-hour) and to output a prediction of a hypoglycemic risk occurrence in the next overnight period (e.g., 6 hours). The 1-D CNN model can include a combination of convolutional layers, activation layers, average pooling layers, and fully-connected layers. The integration of convolutional layers, activation layers, and average pooling layers enhances the predictive accuracy of overnight hypo risk prediction model 8804. Convolutional layers efficiently process spatial and temporal information in order to extract features, activation layers introduce necessary non-linearities for modeling complex patterns, and average pooling layers reduce overfitting and computational complexity.

In some embodiments, the 1-D CNN model for overnight hypo prediction is a neural network consist of layers, including convolutional layers, ReLU activation layers, average/max pooling layers, and fully-connected layers. As one example, the 1-D CNN model, which takes a glucose trace covering a 24-hour period and predicts hypo risk occurrence in the next 6-hour overnight period, can be implemented with at least 3 Convolutional Layers, at least 3 ReLU Activation Layers, at least 1 Average Pooling Layer, and at least 2 Fully-Connected Layers. In this example, the overall neural network of overnight hypo risk prediction model 8804 can be implemented with a feed forward design to extract and aggregate features from the glucose trace, in a progressive fashion. Each layer can be constructed particularly, e.g. length of digital filters, step size, etc., to be effective for operations at a specific time scale. As a non-limiting example, overnight hypo risk prediction model 8804 can be implemented with around 16K parameters, and with a structure for analyzing a large variety of features that are potentially relevant to the occurrence of hypoglycemic conditions. In a particular example, overnight hypo risk prediction model 8804 can be trained using a predetermined number of days (e.g., 4000) worth of glucose traces from a number of different patients (e.g., 80 patients) to optimize values of the 16K model parameters.

Similar designs of 1-D CNN models can be constructed to take different lengths or forms of glucose trace data, such as for a duration of 36 hours or 48 hours. This can result in more accurate prediction of overnight hypo occurrences for certain diabetic populations. In any event, the lengths and forms of data used to train the 1-D CNN model corresponds to the lengths or forms of data that can be input into the 1-D CNN model. Such 1-D CNN models can have different constructions of the fundamental Neural Network layers (i.e. Convolutional Layers, Activation Layers, Fully-Connected Layers, etc.), however they are based on the same ML technique and also share a very similar Neural Network topology. Convolutional layers excel in extracting hierarchical features from sequential data. In CGM data, these features might include the rate of glucose change, the effect of meals, exercise, and insulin administration over time, which are critical for accurate predictions. Activation layers such as ReLU (Rectified Linear Unit) or leaky ReLU are used to introduce non-linearities in the model, allowing it to learn complex and non-linear relationships inherently present in physiological data. Average pooling layers are used to reduce the spatial dimensions of the CGM data being processed by the model, which can translate to temporal dimension reduction.

In other embodiments, alternative approaches of deep learning-based ML model development can be used, such as recurrent neural network (CNN) based ML algorithms, such as Long Short Term Memory (LSTM) models.

### Personalization of the Model(s) During the Personalization Phase

The overnight prediction performance of overnight hypo risk prediction model 8804 can be further improved by training to learn unique characteristics of each individual, i.e., by personalization of the ML models. This can be achieved at three different levels, prediction threshold personalization, feature personalization and developing personalized ML model for each person.

For prediction threshold personalization, the overnight hypoglycemia prediction results could have person-wise systematic bias, i.e., either over-predicting (too many false positive) or under-predicting (too many false negative) for a given person when using the overnight prediction threshold that was optimized at the population level. This type of personal level bias can be mitigated by introducing an adaptive personalized adjustment to the overnight prediction threshold. The adjustment can be implemented using an adaptive parameter to the overnight prediction threshold. This adjustment can be applied to a population level prediction threshold and compared with a predicted probability of overnight hypoglycemia. If the predicted probability of overnight hypoglycemia is greater than the combination of the population level prediction threshold and the adjustment, then overnight hypo risk prediction model 8804 can predict that hypoglycemia will take place (positive prediction); otherwise, overnight hypo risk prediction model 8804 can predict that hypoglycemia will NOT take place (negative prediction). The adjustment value can be periodically updated based on overnight prediction performance.

Training overnight hypo risk prediction model 8804 can include assessing the overnight prediction results. The overnight prediction performance of overnight hypo risk prediction model 8804 should be assessed based on the untreated events available by the time of the assessment. An untreated event can be either a negatively predicted event or a positively predicted event that were know that the user did take preventive actions based on the overnight prediction. True and false negative predictions can be detected from the negatively predicted events, while the true and false positive events can be detected from the untreated positively predicted events. A true positive can be considered when hypoglycemia took place in a positively predicted untreated event; a false positive can be when hypoglycemia did not take place in a positively predicted untreated event; a true negative can be when hypoglycemia did not take place in a negatively predicted event; and a false negative can be when hypoglycemia took place in a negatively predicted event.

Based on the overnight prediction results and glycemic metrics of a patient, overnight hypo risk prediction model 8804 can be reevaluated periodically (e.g., bi-weekly) assessment of whether the overnight hypoglycemia is over-predicted by the ML model. For example, the reevaluation can assess whether the time below range of the glucose value is acceptable by a standardized value (e.g., provided by the standard care of the American Diabetes Association (ADA)) and the false positive rate is higher than the threshold (e.g., 0.5 per week), then the overnight prediction results can be assessed as over predicted.

Personalization of overnight hypo risk prediction model 8804 for overnight hypoglycemia detection can be achieved by rescaling glucose values (G) using the median (M) and absolute median deviation (MAD) prior to feature extraction. For example, the normalized glucose value after rescaling (Gn) can be as indicated by:

$${G}_{n} = \frac{G - M}{MAD}$$

In some embodiments, the disclosure contemplates training overnight hypo risk prediction model 8804 using the data from the entire population. The personalization is reflected in two aspects, 1) the model is trained by the features that were extracted after personalized glucose value linear transformation and 2) the overnight prediction is also using the features that were extracted after rescaling the glucose values using the M and MAD of the person for whom the overnight prediction is performed.

In some embodiments, personalization of the overnight prediction can be achieved by developing overnight hypo risk prediction model 8804 for each person using the data collected from the same person or a cohort associated with that person. Overnight hypo risk prediction model 8804 overcomes two technical barriers for developing and deploy personalized model for each user: 1) the time it takes to collect enough data for developing the model and 2) the cost for training and deploying a personally developed ML model for each user. For this purpose, the disclosure contemplates knowledge about the amount of the data required to train an individual model that can outperform a population model. The data amount requirement can be learned by building personalized models using various amount of data, and subsequently examining the overnight prediction performance metrics as a function of the data amount. The disclosure will determine the required data size as such that the median prediction performance metrics of the personalized ML models are the same as the population-based ML model.

In some embodiments, a personalized version of overnight hypo risk prediction model 8804 can gradually replace the population version through period of time. During this period of time, the final prediction probability can be the weighted average of the output from the population and personal versions of overnight hypo risk prediction model 8804, and the weight of the personal model can be gradually increased from 0 to 100%. A separate top-level ML model can be trained to compute the weight of the personal model. This can be a regression model for computing the weight of the personalized version of overnight hypo risk prediction model 8804. It can take the duration of the data collection for the personalized version of overnight hypo risk prediction model 8804 as a feature, in addition to the original features extracted from the glucose data for the overnight prediction. Therefore, this top-level model can learn to adjust the weight of the personalized version of overnight hypo risk prediction model 8804 according to the data size that it trained with, as well as the characteristics of the features of each prediction.

### Hybrid Model Design

In some embodiments, overnight hypo risk prediction model 8804 can be implemented using a hybrid model design with multi-model prediction for improved prediction accuracy (e.g., as an alternative to or in addition to the personalization phase of overnight hypo risk prediction model 8804). In some embodiments, there can be a model selection process for identifying a model for performing the overnight hypo risk detection where the overnight prediction performance of all potential models including ML models developed using different approaches such as feature engineering-based and deep learning-based ML models as well as non-ML based models, can be compared. There can be instances where two or more models are similar in overall prediction performance and where each model has its own subset of input data (i.e., different glucose data) for which its prediction performance is superior to other selected models. In such cases, overnight hypo risk prediction model 8804 can be identified as the best model(s) for each prediction based the features of the instance which improves the overall prediction performance. This task of identifying the best model(s) for each prediction can be performed by a separate selection ML model specifically trained for identifying and selecting models for use as overnight hypo risk prediction model 8804.

As parameter adaptation takes place for each user, information on the distribution and trending of these parameters can be collected and used to update the initial parameter values of systems being deployed to new users. For existing users, parameters belonging to the third category as described in step 2 of the previous section can also be updated based on this continued parameter adaptation learned from all the users.

In some embodiments, the selection ML model can select best model(s) from several models for each prediction. This is a multi-label classification problem, i.e., for each instance it will classify each model whether it should or should not be used for the overnight prediction. The selection ML model can be developed using the same training data used for developing the ML models for the overnight hypoglycemia prediction, after an additional label has been added for each model. The label for each model indicates whether the overnight prediction results of the particular model is correct.

In embodiments involving selection between multiple models, the selection ML model can select (and extract) features based on the available glucose data and pick which model(s) will be best choice for this prediction based on the selected and extracted features. The overnight hypoglycemia risk can then be predicted by the selected model(s). If more than one model is selected, then the final prediction will be based on the average of all selected models. The average of the selected models may be a weighted or unweighted average.

FIG. 9 is a flowchart illustrating the method 900 of dynamically generating graphical user interfaces based on hypo risk predictions. Method 900 can be performed by processing logic that can comprise hardware (e.g., circuitry, dedicated logic, programmable logic, microcode, etc.), software (e.g., instructions executing on a processing device), or a combination thereof. As a non-limiting example, one or more functions described with respect to FIG. 9 can be performed by a data receiving device (e.g., data receiving device 120 of FIG. 1B), multi-purpose data receiving device (e.g., multi-purpose data receiving device 130 of FIG. 1B), a user device (e.g., user device 140 of FIG. 1B) or an overnight hypo risk prediction model (e.g., overnight hypo risk prediction model of FIG. 8). In such an embodiment, any of these components can execute code in memory to perform certain steps of method 900 of FIG. 9. Accordingly, the following discussion of method 900 will refer to components of FIGS. 1B and 8 as an exemplary non-limiting embodiment. Moreover, it is to be appreciated that not all steps can be needed to perform the disclosure provided herein. Further, some of the functions can be performed simultaneously, in a different order, or by the same components than shown in FIG. 9, as will be understood by a person of ordinary skill in the art.

In 9000, a graphical user interface application installed on a mobile device, such as data receiving device 120, receives user analyte data from an in vivo analyte sensor. The in vivo analyte sensor, such as sensor control device 102, in vivo analyte sensor 104 and/or analyte sensor 110, is configured to be attached to a user and configured to provide continuous glucose data to the mobile device. In some embodiments, the user analyte data can include historical analyte data for a predetermined time period (e.g., 24 hours). In some embodiments, the mobile device can retrieve the historical analyte data from memory. The graphical user interface application can be configured to communicate with the in vivo analyte sensor.

In 9002, the graphical user interface application selects a hypo risk prediction model, such as overnight hypo risk prediction model 8804, responsive to a determination that an upcoming time period corresponds to a future overnight time period. In some embodiments, the upcoming time period can be user-defined, i.e., the user can define what constitutes an “overnight” time period. For example, one user can define the overnight time period as 10 pm to 7 am and another user can define the overnight time period as 12 am to 9 am.

The hypo risk prediction model can be personalized to the user associated with the user analyte data provided by the in vivo analyte sensor. In some embodiments, the selection can be triggered based on a user input, such as an interaction with the graphical user interface application. In other embodiments, the hypo risk prediction model can be configured to operate continuously (e.g., every time user analyte data is received from the in vivo analyte sensor such as once per minute). In other embodiments, the hypo risk prediction model can be enabled during a prespecified time period, the defined overnight period.

The user input can include a request for an overnight prediction for the upcoming time period. In some embodiments, the hypo risk prediction model can be selected from one or more hypo risk prediction models. The mobile device can store or otherwise have access to (e.g., stored on a cloud network) the one or more hypo risk prediction models and the one or more of the hypo risk prediction models can be personalized for the user. In some embodiments, the hypo risk prediction model can be a population (or cohort) based model that is associated with the user based on one or more characteristics, such as, age, gender, and medical conditions.

In 9004, the selected hypo risk prediction model generates a predicted hypoglycemic risk associated with the user and based on the user analyte data. In some embodiments the user analyte data includes historical analyte data and continuous glucose data provided by the in vivo analyte sensor (which can also provide the historical analyte data). The primary input to the selected hypo risk prediction model is the continuous glucose data. The primary output is a prediction of a hypoglycemic condition occurring during a future overnight time-period. This condition can be simply defined as glucose levels falling below a particular threshold level. The hypo risk prediction model can also generate a prediction that is a combination of multiple condition likelihoods and conditions. For instance, the hypo risk prediction model can define multiple thresholds (e.g., 70 mg/dL the likelihood must be 90% or more, but at 54 mg/dL the likelihood must be 50% or more).

In 9006, an application on the mobile device modifies a visual element of the graphical user interface application displayed by the mobile device based on the output provided by the hypo risk prediction model and any components (e.g., action engine 8810). The visual element comprises textual elements selected based on the predicted overnight hypo risk. The textual elements can include one or more of a recommendation, an alert, a notification, a visual indication of the prediction, such as a graph and a trend arrow. The recommendation can include actions the patient can take to mitigate the predicted risk, such as consuming food or some form of nutrition, what type of food or nutrition to consume, a quantity of food or nutrition to consume, setting the low glucose alarm, and setting the predicted low alarm. For example, the application can recommend to drink a predetermined food based on the predicted overnight hypo risk such as a sugary beverage (e.g., fruit juice such as apple or orange juice) or to consume a certain amount of carbohydrates. In some embodiments, this recommendation is based on the predicted overnight hypo risk as well as any user data such as their historical glucose data, their current glucose value, glycemic patterns over a predetermined time period, and user medical conditions.

In some embodiments, the visual element visually represents the predicted overnight hypo risk, such as through a number scale, a percentage value, risk level, and/or color coded icons or visual indicators, and any combination thereof.

In some embodiments, the recommendation includes modifications for a user's current medication or insulin dosage. The recommendation can be based on the user's medication. For example, for users on multiple daily injections or basal only therapy, the recommendation may suggest reducing a basal dose amount based on the overnight hypo risk prediction (e.g., a percentage reduction based on the risk determination, a reduction based on risk level such as a slight dose reduction for a low risk level, moderate dose reduction for medium risk level, significant dose reduction for a high risk level). In some embodiments, the suggested modification is for a one-time adjustment (e.g., reduction) of basal dose amount if overnight hypo risk prediction is high. As another example, for automated insulin delivery users, the recommendation may suggest adjusting target glucose as a way to reduce basal infusion rate.

### General

As used herein, the term “user analyte data” comprises glucose data. The data can be time-series glucose data (also referred to herein as a glucose trace), for example representing 24 hours, 36 hours or 48 hours before an overnight period. The user analyte data can optionally include other CGM data and other patient data.

As used herein, the term “mobile device” is synonymous with the data receiving device 120, multi-purpose data receiving device 130 and/or user device 140.

As used herein, the term “in vivo analyte sensor” is synonymous with the sensor control device 102, in vivo analyte sensor 104 and/or analyte sensor 110.

As used herein, the term “overnight period” is a time period commencing at “bed time” and corresponding to when the user is asleep or intending on being asleep. The overnight period can be a predetermined or prespecified time period. The predetermined or prespecified time period can be defined by the number of hours, such as 7 hours, 8 hours or more, and/or can be defined by the start and end time. The overnight period can begin at a predetermined or prespecified start time, for example 10 P.M., in response to input from the user to their mobile device (e.g. input indicating a sleep event), or as instructed by a trained scheduling module. The overnight period can end at a predetermined or prespecified end time, for example 7 A.M., in response to input from the user to the mobile device (e.g. input indicating an awake event), or as instructed by the trained scheduling module.

As used herein, the term “visual element” can include any visual element, including visualization 8808, displayed on the graphical user interface, not limited to graphical and textual elements. Visual elements for the purpose of the present invention include alerts, notifications, recommendations, predictions, detections, information and the like.

As used herein, the term “textual element” is a particular type of visual element and can include any textual (i.e. alphanumeric) element displayed on the graphical user interface.

As used herein, the term “one or more features associated with the user” include features such as user age type (e.g. adult, teenagers, children) and conditions (e.g. diabetes patients, healthy subjects). Other features can be determined from the user's CGM data or other user data.

As used herein, the term “processor” can refer to any processor, including but not limited to processor 222 of data receiving device 120, processor 166 of analyte sensor 110, a processor of remote application server 155, a processor of user device 140, a processor of multi-purpose user receiving device 130, a processor of local computer system 170, and a processor of trusted computer system 180. The term “computing device” can refer to any computing device, not limited to data receiving device 120, multi-purpose data receiving device 130, user device 140, remote application server 155, local computer system 170 and trusted computer system 180.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A method, comprising:
receiving, at a mobile device, user analyte data from an in vivo analyte sensor, wherein the in vivo analyte sensor is configured to be attached to a user;
selecting a hypo risk prediction model responsive to a determination that an upcoming time period corresponds to a future overnight time period;
generating, by the hypo risk prediction model and based on the user analyte data, a predicted hypoglycemic risk associated with the user; and
modifying a visual element of a graphical user interface displayed at the mobile device, wherein the visual element comprises textual elements selected based on the predicted hypoglycemic risk.

2. The method of claim 1, wherein the predicted hypoglycemic risk is associated with the future overnight time period.

3. The method of claim 1, wherein the determination that the upcoming time period corresponds to the future overnight time period is based on a user input received via the graphical user interface.

4. The method of claim 1, wherein the hypo risk prediction model is selected based on one or more features associated with the user.

5. The method of claim 1, wherein the hypo risk prediction model comprises a 1-dimensional convolutional neural network.

6. The method of claim 1, further comprising:
training the hypo risk prediction model to form a personalized hypo risk prediction model based on one or more features associated with the user.

7. The method of claim 1, wherein the hypo risk prediction model comprises two or more trained models, and wherein the predicted hypoglycemic risk is generated based on an average of each output from the two or more trained models.

8. A system comprising:
a memory;
a processor coupled to the memory and configured to:
receive, at a mobile device, user analyte data from an in vivo analyte sensor, wherein the in vivo analyte sensor is configured to be attached to a user;
select a hypo risk prediction model responsive to a determination that a upcoming time period corresponds to a future overnight time period;
generate, by the hypo risk prediction model and based on the user analyte data, a predicted hypoglycemic risk associated with the user; and
modify a visual element of a graphical user interface displayed at the mobile device, wherein the visual element comprises textual elements selected based on the predicted hypoglycemic risk.

9. The system of claim 8, wherein the predicted hypoglycemic risk is associated with the future overnight time period.

10. The system of claim 8, wherein the determination that the upcoming time period corresponds to the future overnight time period is based on a user input received via the graphical user interface.

11. The system of claim 8, wherein the hypo risk prediction model is selected based on one or more features associated with the user.

12. The system of claim 8, wherein the hypo risk prediction model comprises a 1-dimensional convolutional neural network.

13. The system of claim 8, wherein the processor is further configured to:
training the hypo risk prediction model to form a personalized hypo risk prediction model based on one or more features associated with the user;
receiving second user analyte data; and
generating, by the personalized hypo risk prediction model and based on the second user analyte data, a second predicted hypoglycemic risk associated with the user, wherein the second predicted hypoglycemic risk is associated with a second future overnight time period.

14. The system of claim 8, wherein the hypo risk prediction model comprises two or more trained models, and wherein the predicted hypoglycemic risk is generated based on an average of each output from the two or more trained models.

15. A non-transitory computer-readable device having instructions stored thereon that, when executed by a computing device, causes the computing device to perform operations comprising:
receiving, at a mobile device, user analyte data from an in vivo analyte sensor, wherein the in vivo analyte sensor is configured to be attached to a user;
selecting a hypo risk prediction model responsive to a determination that a upcoming time period corresponds to a future overnight time period;
generating, by the hypo risk prediction model and based on the user analyte data, a predicted hypoglycemic risk associated with the user; and
modifying a visual element of a graphical user interface displayed at the mobile device, wherein the visual element comprises textual elements selected based on the predicted hypoglycemic risk.

16. The non-transitory computer-readable device of claim 15, wherein the predicted hypoglycemic risk is associated with the future overnight time period.

17. The non-transitory computer-readable device of claim 15, wherein the determination that the upcoming time period corresponds to the future overnight time period is based on a user input received via the graphical user interface.

18. The non-transitory computer-readable device of claim 15, wherein the hypo risk prediction model is selected based on one or more features associated with the user.

19. The non-transitory computer-readable device of claim 15, wherein the hypo risk prediction model comprises a 1-dimensional convolutional neural network.

20. The non-transitory computer-readable device of claim 15, wherein the hypo risk prediction model comprises two or more trained models, and wherein the predicted hypoglycemic risk is generated based on an average of each output from the two or more trained models.
